(19) United States
(12) Patent Application Publication
(10) Pub. No.: US 2008/0318284 A1
(43) Pub. Date: Dec. 25, 2008

(54) Processes for Producing a Fermentation Product

(75) Inventors: Chee Leong Soong, Raleigh, NC (US); Shiro Fukuyama, Chiba (JP); Jiyin Liu, Raleigh, NC (US)

Correspondence Address: NOVOZYMES NORTH AMERICA, INC., 500 FIFTH AVENUE, SUITE 1600, NEW YORK, NY, 10110, US

(73) Assignee: Novozymes North America, Inc., Franklinton, NC (US)

(21) Appl. No.: 12/094,048

(22) PCT Filed: Dec. 20, 2006
(86) PCT No.: PCT/US2006/062379

§ 371 Date: May 16, 2008

Related U.S. Application Data
(60) Provisional application No. 60/752,841, filed on Dec. 22, 2005.
(60) Provisional application No. 60/797,756, filed on May 4, 2006.

(51) Int. Cl. C12P 19/20

(52) U.S. Cl. 435/96

(57) ABSTRACT

The present invention relates to processes for producing a fermentation product comprising (a) saccharifying starch-containing material below the initial gelatinization temperature in the presence of i) from 0.001-50 AGU/g DS, preferably 0.01 to 10 AGU/g DS alpha-glucosidase activity more than the native amount of endogenous alpha-glucosidase present in the starch-containing material, and ii) from 0 (zero) to 10 FAU-F/g DS of alpha-amylase activity, and (b) fermenting using a fermenting organism. The invention also relates to an enzymatic composition for use in a process of the invention.

### REFERENCE TO A SEQUENCE LISTING

This application contains a Sequence Listing in computer readable form. The computer readable form is incorporated herein by reference.

### FIELD OF THE INVENTION

The present invention relates to processes for production of a fermentation product from starch-containing material, such as granular starch, at a temperature below the initial gelatinization temperature of the starch-containing material. The invention also relates to an enzymatic composition and the use thereof in a process of the invention.

### BACKGROUND OF THE INVENTION

Grains, cereals or tubers of plants contain starch. The starch is in the form of microscopic granules, which are insoluble in water at room temperature. When an aqueous starch slurry is heated) the granules swell and eventually burst, dispersing the starch molecules into the solution. During this “gelatinization” process: there is a dramatic increase in viscosity. Because the solids level in a typical industrial process is around 30-40%, the starch has to be thinned or “liquefied” so that it can be handled. This reduction in viscosity is generally accomplished by enzymatic degradation in a process referred to as liquefaction. During liquefaction, the long-chained starch is degraded into smaller branched and linear chains of glucose units (dextrins) by an alpha-amylase.

A conventional enzymatic liquefaction process may be carried out as a three-step hot slurry process. The slurry is heated to between 80-85° C. and thermostable alpha-amylase added to initiate liquefaction. The slurry is then jet-cooked at a temperature between 105-125° C. to complete gelatinization of the slurry, cooled to 60-95° C. and, generally, additional alpha-amylase is added to finalize hydrolysis. The liquefaction process is generally carried out at a pH between 5 and 6. Milled and liquefied whole grains are known as mash.

During saccharification, the dextrins from the liquefaction are further hydrolyzed to produce low molecular sugars (DP₁₋₃) that can be metabolized by a fermenting organism, such as yeast. The hydrolysis is typically accomplished using glucoamylase, alternatively or in addition to glucoamylases, alpha-glucosidase and/or acid alpha-amylases can be used. A full saccharification step typically lasts up to 72 hours, however it is common only to do a pre-saccharification of, e.g., 40-90 minutes at a temperature above 50° C., followed by a complete saccharification during fermentation in a process known as simultaneous saccharification and fermentation (SSF).

Fermentation is performed using a fermenting organism, such as yeast, which is added to the mash. Then the fermentation product is recovered. For ethanol, e.g., fuel, portable, or industrial ethanol, the fermentation is carried out, for typically 35-60 hours at a temperature of typically around 32° C. When the fermentation product is beer, the fermentation is carded out, for typically up to 8 days at a temperature of typically around 14° C.

Following fermentation, the mash may be used, e.g., as a beer, or distilled to recover ethanol. The ethanol may be used as, e.g. fuel ethanol, drinking ethanol, and/or industrial ethanol.

It will be apparent from the above discussion that the starch hydrolysis in a conventional process is very energy consuming due to the different temperature requirements during the various steps.

U.S. Pat. No. 4,316,956 provides a fermentation process for conversion of granular starch into ethanol.

European Patent No. EP 140410-A provides an enzyme composition for starch hydrolysis.

WO 2004/081193 concerns a method of producing high levels of alcohol during fermentation of plant material. The method includes i) preparing the plant material for saccharification, ii) converting the prepared plant material to sugar without cooling, and iii) fermenting the sugars.

WO 2004/0106533 concerns a process of producing an alcohol product from granular starch comprising a pre-treatment at an elevated temperature below the initial gelatinization temperature of said granular starch followed by simultaneous saccharification and fermentation. The process is performed in the presence of an acid alpha-amylase activity, a maltose generating enzyme activity and an alpha-glucosidase.

The object of the present invention is to provide improved processes for conversion of starch-containing material, such as granular starch, into a fermentation product, such as ethanol.

### SUMMARY OF THE INVENTION

This present invention relates to processes of producing a fermentation product from starch-containing materials (e.g., fractionated starch-containing material). A process of the invention includes simultaneously or sequentially saccharification and fermentation steps carried out at a low temperature.

In the first aspect the invention relates to a process for producing a fermentation product from starch-containing material comprising:

(a) saccharifying starch-containing material in the presence of:
i) from 0.001-50 AGU/g DS, preferably 0.01 to 10 AGU/g DS of alpha-glucosidase more than the amount of alpha-glucosidase present endogenously in the starch-containing material, and
ii) from above 0 (zero) to 10 FAU-F/g DS of alpha-amylase, at a temperature below the initial gelatinization temperature of said starch-containing material,

(b) fermenting using a fermenting organism.

In an embodiment the invention relates to a process for producing a fermentation product from starch-containing material derived from a modified plant comprising:

(a) saccharifying starch-containing material below the initial gelatinization temperature in the presence of
i) alpha-glucosidase activity, and
ii) from above 0 (zero) to 10 FAU-F/g DS of alpha-amylase activity,

(b) fermenting using a fermenting organism,

wherein the amount of alpha-glucosidase in step (a) is higher that the native amount of endogenous alpha-glucosidase in corresponding unmodified starch-containing plant material.

In a second aspect the invention relates to a process for producing a fermentation product from starch-containing material comprising the steps of:

(a) liquefying starch-containing material in the presence of an alpha-amylase;

(b) saccharifying the liquefied material obtained in step (a) at a temperature in the range from 20-60° C. in the presence of:
i) from 0.001-50 AGU/g DS, preferably 0.01 to 10 AGU/g DS alpha-glucosidase more than the native amount of endogenous alpha-glucosidase present in the starch-containing material, and
ii) from above 0 (zero) to 10 FAU-F/g DS of alpha-amylase.

(c) fermenting using a fermenting organism.

In an embodiment the invention relates to a process for producing a fermentation product from starch-containing material derived from a modified plant comprising:

(a) liquefying starch-containing material in the presence of an alpha-amylase;

(b) saccharifying starch-containing material below the Initial gelatinization temperature in the presence of
i) alpha-glucosidase activity, and optionally
ii) from 0 (zero) to 10 FAU-F/g DS of alpha-amylase activity,

(c) fermenting using a fermenting organism.

wherein the amount of alpha-glucosidase in step (a) is higher that the native amount of endogenous alpha-glucosidase in corresponding unmodified starch-containing plant material.

In a third aspect the invention relates to a composition comprising an alpha-glucosidase and an alpha-amylase.

In a fourth aspect the invention relates the use of a composition of the invention.

### BRIEF DESCRIPTION OF THE DRAWING

FIG. 1 shows the maltose generation when hydrolyzing corn starch with corn alpha-glucosidase combined with Alpha-Amylase A.

FIG. 2 shows the glucose generation when hydrolyzing corn starch with corn alpha-glucosidase combined with Alpha-Amylase A.

FIG. 3 shows the ethanol yields for one-step fermentation processes where ground corn is subjected to different concentrations of corn alpha-glucosidase and Alpha-Amylase A.

FIG. 4 shows the pH stability of corn alpha-glucosidase compared to rice alpha-glucosidase.

FIG. 5 shows the temperature stability of corn alpha-glucosidase compared to rice alpha-glucosidase.

FIG. 6 shows the stability of corn alpha-glucosidase compared to rice alpha-glucosidase at a ethanol concentration 20 vol. %.

FIG. 7 compares the performance (ethanol g/l) of:

1) Alpha-glucosidase from corn (2.6 AGU/g DS);

2) Alpha-amylase A (0.127 FAU-F/g DS;

3) Alpha-amylase A (0.127 FAU-F/g DS and Glucoamylase TC (0.34 AGU/g DS;

4) Alpha-glucosidase from corn (2.6 AGU/g DS) Alpha-amylase A (0.127 FAU-F/g DS) and Glucoamylase TC (0.34 AGU/g DS;

in a one-step simultaneous saccharification and fermentation process (SSF).

FIG. 8 compares the performance (ethanol g/l) of

1) Alpha-amylase A (0.057 FAU-F/g DS and Glucoamylase AN (1.0 AGU/g DS)

2) Alpha-glucosidase from corn (2.6 AGU/g DS), Alpha-amylase A (0.057 FAU-F/g DS), and Glucoamylase AN (1.0 AGU/g DS),

in a one-step simultaneous saccharification and fermentation process (SSF).

FIG. 9 compares the performance (Ethanol g/l) of:

1) Alpha-amylase A (0.0.57 FAU-F/g DS and Glucoamylase SF (1.68 AGU/g DS);

2) Alpha-glucosidase from corn (2.6 AGU/g DS), Alpha-amylase A (0.57 FAU-F/g DS and Glucoamylase SF (1.68 AGU/g DS);

in a one-step simultaneous saccharification and fermentation process (SSF).

FIG. 10 compares the performance (Ethanol g/l) of:

1) Alpha-amylase A (0.57 FAU-F/g DS) and Glucoamylase TC (0.34 AGU/g DS,

2) Alpha-amylase A (0.57 FAU-F/g DS), Glucoamylase TC (0.34 AGU/g DS, and alpha-glucosidase from Bacillus stearothermophilus (10 units/g DS);

3) Alpha-amylase A (0.57 FAU-F/g DS), Glucoamylase TC (0.34 AGU/g DS), and alpha-glucosidase from yeast (25 units/g DS)

in a one-step simultaneous saccharification and fermentation process (SSF).

### DETAILED DESCRIPTION OF THE INVENTION

This present invention relates to processes of producing a fermentation product from starch-containing material (e.g., fractionated starch-containing material). The amount of native endogenous enzyme active in starch-containing plant material, at the time of initiating production of a desired fermentation product, depends to a large extent on the quality of the harvested starch-containing plant material and the post-harvest handling of the plant material. For instance, if the starch-containing plant material is dried and/or stored for a long period of time some if not all endogenous enzymatic activity may have disappeared. The present invention deals with this problem. Native endogenous corn alpha-glucosidase was found to be present in ground corn (without any other treatment) in amounts corresponding to enzymatic activity levels as high as from 1 to 2 AGU/g DS (see Example 3). The inventors found that the actual total amount of plant alpha-glucosidase present during simultaneous saccharification and fermentation (SSF) of uncooked starch-containing plant material has a significant impact on the final fermentation yield. The inventors also found that the fermentation yield may be increased by adding more alpha-glucosidase than present natively in the starch-containing plant material. In Example 5 it is shown that when adding 1.13 AGU/g DS, 2.25 AGUig DS, and 4.51 AGU/g DS of corn alpha-glucosidase during SSF in combination with alphaamylase (which is usually added during SSF of uncooked starch-containing material) the ethanol yield is increased significant. The inventors have also found that the fermentation yield may be increased further by selecting certain combinations of alpha-glucosidase and alpha-amylase.

Therefore, in the first aspect the present invention relates to a process for producing a fermentation product from starch-containing material comprising:

(a) saccharifying starch-containing material below the initial gelatinization temperature in the presence of
i) from 0.001-50 AGU/g DS, preferably 0.01 to 10 AGU/g DS alpha-glucosidase activity more than the native amount of endogenous alpha-glucosidase present in the starch-containing material, and
ii) from above 0 (zero) to 10 FAU-F/g DS of alpha-amylase activity,

(b) fermenting using a fermenting organism.

It should be understood that the higher amounts of alpha-glucosidase may in one embodiment be provided by using a plant material modified in order to contain a higher amount of alpha-glucosidase compared to starch-containing plant material derived from unmodified plants.

It should also be understood that other enzyme activities, such as glucoamylase and/or alpha-amylase activity, may also be provided to a process of the invention by modifying the plant material to express said enzyme activities. Means for modifying plant material are well know in the art. How to express alpha-glucosidase and other enzyme activities in transgenic plants is described further below in the “Expression of alpha-glucosidase in transgenic plants”.

In such case the invention relates to process for producing a fermentation product from starch-containing material derived from a modified plant comprising:

(a) saccharifying starch-containing material below the initial gelatinization temperature in the presence of:
i) alpha-glucosidase activity, and
ii) from above 0 (zero) to 10 FAU-F/g DS of alpha-amylase activity,

(b) fermenting using a fermenting organism,

wherein the amount of alpha-glucosidase in step (a) is higher that the native amount of endogenous alpha-glucosidase in corresponding unmodified starch-containing plant material.

In a preferred embodiment the alpha-glucosidase activity amount is in the range from 0.001-50 AGU/g DS, preferably 0.01 to 10 AGU/g DS above the native amount of endogenous alpha-glucosidase in corresponding unmodified starch-containing material. The modified starch-containing plant material may be derived from a transgenic plant. A transgenic plant may be prepared using techniques well know in the art. Examples are described in the “Expression of alpha-glucosidase in transgenic plants” section below. According to this embodiment of the invention the transgenic plant material has a higher amount of endogenous alpha-glucosidase activity compared to the native amount of endogenous alpha-glucosidase in corresponding unmodified starch-containing plant material.

In an embodiment the fermentation product is recovered after fermentation. Step (a) and (b) may be carried out sequentially or simultaneously.

Before step (a), a slurry of starch-containing material, such as granular starch, having 20-55 wt. % dry solids of starch-containing material, preferably 25-45 wt. % dry solids, more preferably 30-40 wt. % dry solids or 30-45 wt. %, may be prepared. The slurry may include water and/or process waters, such as stillage (backset), scrubber water, evaporator condensate or distillate, side stripper water from distillation, or other fermentation product plant process water. Because the process of the invention is carried out below the gelatinization temperature and thus no significant viscosity increase takes place high levels of stillage may be used if desired. In an embodiment the aqueous slurry contains from about 1 to about 70 vol. % stillage, preferably 15-60 vol. % stillage, especially from about 30 to 50 vol. % stillage.

After being subjected to a process of the invention at least 85%, at least 86%, at least 87%, at least 88%, at least 89%, at least 90%, at least 91%, at least 92%, at least 93%, at least 94%, at least 95%, at least 96%, at least 97%, at least 98%, or preferably at least 99% of the dry solids of starch-containing material is converted into a soluble starch hydrolyzate.

In a preferred embodiment step (a) and step (b) are carried out as a simultaneous saccharification and fermentation process. In such preferred embodiment the process is typically carried at a temperature between 25° C. and 40° C., preferably 28° C. and 36° C., such as between 28° C. and 35° C., such as between 28° C. and 34° C., such as around 32° C. According to the invention the temperature may be adjusted up or down during fermentation.

In an embodiment simultaneous saccharification and fermentation is carried out so that the sugar level, such as glucose level, is kept at a low level such as below about 6 wt. %, preferably below about 3 wt. %, preferably below about 2 wt, %, more preferred below about 1 wt. %, even more preferred below about 0.5%, or even more preferred below about 0.25 wt. %. Such low levels of sugar can be accomplished by simply employing adjusted quantities of enzyme and fermenting organism. A skilled person in the art can easily determine which quantities of enzyme and fermenting organism to use. The employed quantities of enzyme and fermenting organism may also be selected to maintain low concentrations of maltose in the fermentation broth. For instance, the maltose level may be kept below about 0.5 wt. % or below about 0.2 wt. %.

The process of the invention may be carried out at a pH in the range between 3 and 7, preferably from 3 to 6, or more preferably from 3.5 to 5.0.

### Starch-Containing Materials

Any suitable starch-containing plant material, including granular starch, may be used according to the present invention. The starting material is generally selected based on the desired fermentation product. Examples of starch-containing starting materials, suitable for use in the processes of present invention, include tubers, roots, stems, whole grains, corns, cobs, wheat, barley, rye, sago, cassava, tapioca, sorghum, nice peas, beans, sweet potatoes, or mixtures thereof, or cereals, sugar-containing raw materials, such as molasses, fruit materials, sugar cane or sugar beet, potatoes, and cellulose-containing materials, such as wood or plant residues. Contemplated are both waxy and non-waxy types of corn and barley.

The term “granular starch” means raw uncooked starch, i.e. starch in its natural form found in cereal, tubers or grains. Starch is formed within plant cells as tiny granules insoluble in water. When put in cold water, the starch granules may absorb a small amount of the liquid and swell. At temperatures up to 50° C. to 75° C. the swelling may be reversible. However, with higher temperatures an irreversible swelling called “gelatinization” begins. Granular starch to be processed may be a highly refined starch quality, preferably at least 90%, at least 95%, at least 97% or at least 99.5% pure or it may be a more crude starch containing material comprising, e.g., milled whole grain including non-starch fractions such as germ residues and fibers.

The term “initial gelatinization temperature” means the lowest temperature at which gelatinization of the starch commences. Starch heated in water begins to gelatinize between 50° C. and 75° C.; the exact temperature of gelatinization depends on the specific starch, and can readily be determined by the skilled artisan. Thus, the initial gelatinization temperature may vary according to the plant species, to the particular variety of the plant species as well as with the growth conditions. In the context of this invention the initial gelatinization temperature of a given starch-containing material is the temperature at which birefringence is lost in 5% of the starch granules using the method described by Gorinstein S. and Lii. C., Starch/Stärke, Vol. 44 (12), pp. 461-466 (1992).

### Fractionation of Starch-Containing Material

In an embodiment the starch-containing plant material is fractionated into one or more components, including fiber, germ, and a mixture of starch and protein (endosperm). Fractionation may according to the invention be done using any suitable technology or apparatus. For instance, Satake has manufactured a system suitable for fractionation of plant material such as corn.

The germ and fiber components may be fractionated from the remaining portion of the endosperm. In an embodiment of the invention the starch-containing material is plant endosperm, preferably corn endosperm. Further the endosperm may be reduced in particle size and combined with the larger pieces of the fractionated germ and fiber components for fermentation.

Fractionation can be accomplished, e.g., using the apparatus disclosed in US patent application no. 2004/0043117 (hereby incorporated by reference). Suitable methods and apparatus for fractionation include a sieve, sieving and elutriation. Suitable apparatus also include friction mills, such as rice or grain polishing mills (e.g., those manufactured by Satake, Kett, or Rapsco).

### Reducing the Particle Size of Starch-Containing Plant Material

The starch-containing plant raw material, such as whole grain, used in a process of the invention, may preferably be reduced in particle size in order to open up the structure and allowing for further processing. This may be done by milling. Two milling processes are preferred according to the invention: wet and dry milling. In dry milling whole kernels are milled and used. Wet milling gives a good separation of germ and meal (starch granules and protein) and is often applied at locations where the starch hydrolyzate is used in production of syrups. Both dry and wet milling is well known in the art of starch processing and is equally contemplated for the process of the invention. Examples of other contemplated technologies for reducing the particle size of the starch-containing plant material include emulsifying technology and rotary pulsation.

The starch-containing material may be reduced in particle size to between 0.05 to 3.0 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen.

### Fermentation Product

The term “fermentation product” means a product produced by a process including a fermentation step using a fermenting organism. Fermentation products contemplated according to the invention include alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes, vitamins (e.g., riboflavin, B₁₂, beta-carotene); and hormones. In a preferred embodiment the fermentation product is ethanol, e.g., fuel ethanol; drinking ethanol, i.e., portable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferred fermentation processes used include alcohol fermentation processes, as are well known in the art. Preferred fermentation processes are anaerobic fermentation processes, as are well known in the art.

### Fermenting Organism

“Fermenting organism” refers to any organism, including bacterial and fungal organisms, suitable for use in a fermentation process and capable of producing desired a fermentation product. Especially suitable fermenting organisms are able to ferment, i.e., convert, sugars, such as glucose or maltose, directly or indirectly into the desired fermentation product. Examples of fermenting organisms include fungal organisms, such as yeast. Preferred yeast includes strains of the Saccharomyces spp., and in particular, Saccharomyces cerevisiae. Commercially available yeast include, e.g., Red Star™/Lesaffre Ethanol Red (available from Red Star/Lesaffre, USA) FALI (available from Fleischmann's Yeast, a division of Sums Philp Food Inc., USA), SUPERSTART (available from Alltech), GERT STRAND (available from Gert Strand AB, Sweden) and FERMIOL (available from DSM Specialties).

### Enzymes

### Alpha-Glucosidase

According to the invention any alpha-glucosidase (including enzymes classified as EC 3.2.1.20 or EC 3.2.1.48) may be used according to the invention. Examples of alpha-glucosidases contemplated according to the invention include those derived from microorganisms, such as bacteria and fungi, including yeast and filamentous fungi, Actinomycetes, and plants. In an embodiment the alpha-glucosidase is an acid alpha-glucosidase. This means that the pH optimum is below 7.0, preferably between pH 3-7.

In a preferred embodiment the alpha-glucosidase is stable in the presence of the fermentation product in question at concentrations below 10 vol. %, preferably below 12 vol. %, more preferably below 15 vol, %, more preferably below 18 vol. %, more preferably below 20 vol, %, more preferably below 25 vol. % fermentation product. In a specific embodiment the alpha-glucosidase is stable in the presence of ethanol, preferably at concentrations below 10 vol, %, preferably 12 vol. %, more preferably below 15 vol. %, more preferably below 18 vol. %, even more preferably below 20 vol. %, even more preferably below 25 vol. % ethanol. The ethanol stability may be determined as ethanol stability at the condition described in Example 6. This means that the relative activity is above 50%, preferably above 70%, more preferably above 90% after 10 minutes, preferably after 30 minutes, more preferably after 60 minutes incubation at 30-40° C., preferably at around 37° C.

Bacterial alpha-glucosidases include those derived from a strain of the genus Bacillus, such as a strain of Bacillus stearothermophilus. A commercial Bacillus stearothermophilus alpha-glucosidase is available from Sigma (Sigma cat. No. G3651).

Fungal alpha-glucosidases include those derived from yeast or filamentous fungi. Examples of alpha-glucosidases derived from yeast include those derived from a strain of Candida sp, such as Candida edax, preferably CBD 6451, or from a strain of Saccharomyces, preferably Saccharomyces cerevisae. Other alpha-glucosidases derived from yeast include those derived from Pichia sp., such as Pichia amylophila, Pichia missisipiensis, Pichia wiherhamii and Pichiarhodanensis.

Alpha-glucosidases derived from filamentous fungi, include those from the genus Aspergillus, Fusarium, Mucor, and Penicillium.

Examples of alpha-glucosidases from a strain of Aspergillus include those derived from Aspergillus nidulans (Kato et al., 2002, Appl. Environ Microbiol. 68: 1250-1256), Aspergillus fumigatus (Rudick and Elbein, 1974, Archives of Biochemistry and Biophysics 161: 281-290), Aspergillus flavus (Olutiola, 1981, Mycologia 73: 1130), Aspergillus nidulans (Kato et al., 2002, Appl. Environ. Microbiol. 68: 1250-1256), Aspergillus niger (Rudick et al., 1979, Archives of Biochemistry and Biophysics 193: 509 and Nakamura et. al., 1997, J. Biotechnol 53: 75-84), Aspergillus oryzae (Minetoki et al., 1995, Biosci. Biotech. Biochem 59: 1516-1521, Leibowitz and Mechlinski, 1926, Hoppe-Seyler's Zeitschrift für Physiologische Chemie 154: 64) and Aspergillus fumigatus (US publication no. 2006/0008879). Known alpha-glucosidases also include those derived from a strain of Rhizobium sp. (Berthelot et al., 1999, Appl. Environ Microbiol. 65: 2907-2911), Mucor javanicus (Yamasaki et al., 1978, Berichte des Ohara Instituts für Landwirtschaftiiche Biologie 17: 123), Mucor racemosus (Yamasaki et al., 1977, Agricultural and Biological Chemistry 41: 1553), Mucor rouxii (Flores-Carreon and Ruiz-Herrera, 1972, Biochemica et Biophysica Acta 258: 496), Penicillium pupurogenum (Yamasaki et al., 1976, Agricultural and Biological Chemistry 40: 669), and Penicillium oxalicum (Yamasaki et al., 1977, Agricultural and Biological Chemistry 41: 1451) and Fusarium venenatum (US publication no. 2006/0156437).

In a preferred embodiment the fungal alpha-glucosidase is derived from a strain of the genus Aspergillus, including A. nidulans, A. niger. A. oryzae and A. fumigatus.

In a preferred embodiment the alpha-glucosidase is a plant alpha-glucosidase. The plant alpha-glucosidase may be derived from any plant material, preferably a plant selected from corn (maize), cobs, wheat, barley, rye, milo, sago, cassava, tapioca, sorghum, rice, peas, or beans, sweet potatoes, or a mixture thereof. In a preferred embodiment the alpha-glucosidase is derived from corn.

When using the term “endogenous (plant) alpha-glucosidase” it means alpha-glucosidase enzyme natively produced by the plant in question, such as corn. It is to be understood that according to the invention a plant alpha-glucosidase may be cloned from the plant in question and expressed recombinantly in a suitable host cell using techniques well known in the ad. Alternatively the plant alpha-glucosidase may be purified from the plant in question before being used in a process of the invention. Purification of endogenous corn alpha-glucosidase is described in Examples 1 and 2 below. Further, according to the invention it is also contemplated to increase the alpha-glucosidase activity in a process of the invention by modifying the native plant. This may be done by preparing a transgenic plant expressing increased amounts of alpha-glucosidase. Preparing a transgenic plant capable of expressing increased amounts of alpha-glucosidase can be accomplished by the skilled person in the art using methods well known in the art. The alpha-glucosidase encoding gene may be any alpha-glucosidase encoding gene, preferably of plant, especially corn, origin. However, also alpha-glucosidase genes of bacterial and fungal origin are contemplated. Suitable examples are disclosed in this section.

Contemplated are also alpha-glucosidases which exhibit a high identity to any of above mention alpha-glucosidases, i.e., more than 70%, more than 75%, more than 80%, more than 85% more than 90%, more than 95%, more than 96%, more than 97%, more than 98%, more than 99% or even 100% identity to the mature enzymes sequences.

According to the invention from 0.01 to 10 AGU/g DS of alpha-glucosidase activity is added. In a preferred embodiment from 0.1 to 8 AGU/g DS, preferably 1 to 6 AGU/g DS plant alpha-glucosidase activity more than the native amount present in the starch-containing plant material is present during saccharification or simultaneous saccharification and fermentation (i.e., step (a)). According to the invention the total amount of plant alpha-glucosidase activity present, i.e., endogenous plant alpha-glucosidase activity and added plant alpha-glucosidase activity, may be from above 1 or 2 to 12 AGU/g DS, such as 3 to 10 AGU/g DS, preferable from 4 to 8 AGU/g DS.

### Expression of Alpha-Glucosidase in Transgenic Plants

As mentioned above the amount of alpha-glucosidase in a process of the invention may be increased to the specified amounts by preparing a transgenic plant expressing increased amounts of alpha-glucosidase. It should also be understood that other enzyme activities, including starch-degrading enzyme activities, such as glucoamylase and/or alpha-amylase activity, may also be provided to a process of the invention by modifying the plant material to express said enzyme activities.

A DNA sequence(s) encoding (an) enzyme(s), such as alpha-glucosidase, may be transformed and expressed in transgenic plants using well known techniques, e.g., as described below. The enzyme, preferably alpha-glucosidase may be heterologous or homologous to the plant in question, especially corn.

The transgenic plant may be prepared from any plant comprising starch-containing material. Examples of such are listed in the “Starch-containing materials” section above, and include cereals, such as wheat, oats, rye, barley, rice, sorghum and especially maize (corn).

The alpha-glucosidase is preferably expressed in at least the seeds, preferably corn kernels, such as, e.g., the embryo, endosperm, aleurone and/or seeds coat.

The transgenic plant or plant cell, used in a process of the invention, expressing the alpha-glucosidase may be constructed in accordance with methods known in the art. In short the plant or plant cell is constructed by incorporating one or more expression constructs encoding the alpha-glucosidase into the plant host genome and propagating the resulting modified plant or plant cell into a transgenic plant or plant cell.

Conveniently, the expression construct is a DNA construct which comprises a gene encoding the enzyme in question, preferably alpha-glucosidase, in operable association with appropriate regulatory sequences required for expression of the gene in the plant. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences is determined, e.g., on the basis of when, where and how the enzyme is desired to be expressed. For instance, the expression of the gene encoding alpha-glucosidase may be constitutive or inducible, or may be developmental, stage or tissue specific, and the gene product may be targeted to a specific cell compartment, tissue or plant part such as seeds or leaves. Regulatory sequences are, e.g. described by Tague et al., 1988, Plant, Phys., 86: 506.

For constitutive expression the 35S-CaMV, the maize ubiquitin 1 and the rice actin 1 promoter may be used (Franck et al., 1980, Cell 21, 285-294, Christensen A H, Sharrock R A and Quail 1992. Maize polyubiquitin genes: structure, thermal perturbation of expression and transcript splicing, and promoter activity following transfer to protoplasts by electroporation. Plant Mo. Biol. 18, 675-689; Zhang W, McElroy D. and Wu R 1991, Analysis of rice Act1 5′ region activity in transgenic rice plants. Plant Cell 3, 1155-1165). Organ-specific promoters may, e.g., be a promoter from storage sink tissues such as seeds, potato tubers, and fruits (Edwards & Coruzzi, 1990, Annu. Rev. Genet. 24: 275-303), or from metabolic sink tissues such as meristems (Ito et alt, 1994, Plant Mol. Biol. 24: 863-878), a seed specific promoter such as the glutelin, prolamin, globulin or albumin promoter from rice (Wu et al., Plant and Cell Physiology Vol. 39, No. 8 pp. 885-889 (1998)), a Vicia faba promoter from the legumin B4 and the unknown seed protein gene from Vicia faba described by Conrad U. et al, Journal of Plant Physiology Vol. 152% No. 6 pp. 708-711 (1998), a promoter from a seed oil body protein (Chen et al., Plant and Cell Physiology, Vol. 39, No. 9, pp. 935-941 (1998), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., Plant Physiology Vol. 102, No. 3, pp. 991-1000 (1993), the chlorella virus adenine methyltransferase gene promoter (Mitra, A. and Higgins, D W, Plant Molecular Biology Vol. 26, No. 1, pp. 85-93 (1994), or the aldP gene promoter from rice (Kagaya et al., Molecular and General Genetics, Vol. 248, No. 6, pp. 668-674 (1995), or a wound inducible promoter such as the potato pln2 promoter (Xu et al., Plant Molecular Biology Vol, 22, No. 4, pp 573-588 (1993). Likewise, the promoter may inducible by abiotic treatments such as temperature, drought or alterations in salinity or induced by exogenously applied substances that activate the promoter, e.g., ethanol, oestrogens, plant hormones like ethylene, abscisic acid and gibberellic acid and heavy metals.

A promoter enhancer element may be used to achieve higher expression of the enzyme (s) in the plant. For instance, the promoter enhancer element may be an intron which is placed between the promoter and the nucleotide sequence encoding the enzyme. For instance, Xu et al. (Plant Molecular Biology, Vol. 22, No. 4, pp. 573-588 (1993)) discloses the use of the first intron of the rice actin 1 gene to enhance expression.

The selectable marker gene and any other parts of the expression construct may be chosen from those available in the art.

The DNA construct is incorporated into the plant genome according to conventional techniques known in the art, including Agrobacterium-mediated transformation, virus-mediated transformation, micro Injection, particle bombardment, biolistic transformation, and electroporation (Gasser et al., Science, 244: 1293; Potrykus, Bio/Techn. 8: 535 (1990); Shimamoto et al., Nature, 338: 274 (1989)).

Presently, Agrobacterium tumefaciens mediated gene transfer is the method of choice for generating transgenic dicots (for review Hooykas & Schilperoort, 1992, Plant Mol. Biol. 19; 15-38), and can also be used for transforming monocots, although other transformation methods often are used for these plants, Presently, the method of choice for generating transgenic monocots supplementing the Agrobacterium approach is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio/Technology 10: 667-674). An alternative method for transformation of monocots is based on protoplast transformation as described by Omirulleh S, et al., Plant Molecular Biology, Vol 21 No. 3, pp. 415-428 (1993).

Following transformation, the transformants having incorporated the expression construct are selected and regenerated into whole plants according to methods well-known in the art. Often the transformation procedure is designed for the selective elimination of selection genes either during regeneration or in the following generations by using, e.g., co-transformation with two separate T-DNA constructs or site specific excision of the selection gene by a specific recombinase.

### Alpha-Amylase

According to the invention an alpha-amylase may be used in combination with alpha-glucosidase. The alpha-amylase is present in an effective amount present during saccharification and/or fermentation, which include from 0.01 to 3 FAU-F/g DS, preferably from 0.05 to 0.2 FAU-F/g DS.

In a preferred embodiment the alpha-amylase is an acid alpha-amylase, e.g., fungal acid alpha-amylase or bacterial acid alpha-amylase. The term “acid alpha-amylase” means an alpha-amylase (E.C. 3.2.1.1) which added in an effective amount has activity optimum at a pH in the range of 3 to 7, preferably from 3.5 to 6, or more preferably from 4-5.

### Bacterial Alpha-Amylase

According to the invention the bacterial alpha-amylase is preferably derived from the genus Bacillus.

In a preferred embodiment the Bacillus alpha-amylase is derived from a strain of B. licheniformis, S. amyloliquefaciens, B. subtilis or B. stearothermophilus, but may also be derived from other Bacillus sp. Specific examples of contemplated alpha-amylases include the Bacillus licheniformis alpha-amylase shown in SEQ ID NO: 4 in WO 99/19467, the Bacillus amyloliquefaciens alpha-amylase SEQ ID NO; 5 in WO 99/19467 and the Bacillus stearothermophilus alpha-amylase shown in SEQ ID NO: 3 in WO 99/19467 (all sequences hereby incorporated by reference). In an embodiment of the invention the alpha-amylase may be an enzyme having a degree of identity of at least 60%, preferably at least 70%, more preferred at least 80%, even more preferred at least 90%, such as at least 95%, at least 96%, at least 97%, at least 98% or at least 99% to any of the sequences shown in SEQ ID NOS: 1, 2 or 3, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 and 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta (181-182) compared to the wild-type BSG alpha-amylase amino acid sequence set forth in SEQ ID NO: 3 disclosed in WO 99/19467 or deletion of amino acids R179 and G180 using SEQ ID NO: 3 in WO 99/19467 for numbering (which reference is hereby incorporated by reference). Even more preferred are Bacillus alpha-amylases, especially Bacillus stearothermophilus alpha-amylase, which have a double deletion corresponding to delta (181-182) and further comprise a N193F substitution (also denoted I181*+G182*+N193F) compared to the wildtype BSG alpha-amylase amino acid sequence set forth in SEQ ID NO: 3 disclosed in WO 99/19467.

### Bacterial Hybrid Alpha-Amylase

A hybrid alpha-amylase specifically contemplated comprises 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467), with one or more, especially all, of the following substitution:

G48A+T49I+G107A+H156Y+A181T+N190F+1201F+A209V+Q264S (using the Bacillus licheniformis numbering in SEQ ID NO: 4 of WO 99/19467). Also preferred are variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using SEQ ID NO: 5 numbering of WO 99/19467).

### Fungal Alpha-Amylase

Fungal alpha-amylases include alpha-amylases derived from a strain of the genus Aspergillus, such as, Aspergillus oryzae, Aspergillus niger and Aspergillus kawachii alpha-amylases.

A preferred acidic fungal alpha-amylase is a Fungamyl-like alpha-amylase which is derived from a strain of Aspergillus oryzae. According to the present invention, the term “Fungamyl-like alpha-amylase” indicates an alpha-amylase which exhibits a high identity, i.e., more than 70%, more than 75%, more than 80%, more than 85% more than 90%, more than 95%, more than 96%, more than 97%, more than 98%, more than 99% or even 100% identity to the mature part of the amino acid sequence shown in SEQ ID NO: 10 in WO 96/23874.

Another preferred acidic alpha-amylase is derived from a strain Aspergillus niger. In a preferred embodiment the acid fungal alpha-amylase is the one from A. niger disclosed as “AMYA_ASPNG” in the Swiss-prot/TeEMBL database under the primary accession no. P56271 and described in WO 89/01969 (Example 3). A commercially available acid fungal alpha-amylase derived from Aspergillus niger is SP288 (available from Novozymes A/S, Denmark).

Other contemplated wild-type alpha-amylases include those derived from a strain of the genera Rhizomucor and Merpilus, preferably a strain of Rhizomucor pusillus (WO 2004/055178 incorporated by reference) or Meripilus giganteus.

In a preferred embodiment the alpha-amylase is derived from Aspergillus kawachii and disclosed by Kaneko et al., J. Ferment. Bioeng. 81:292-298 (1991) “Molecular-cloning and determination of the nucleotide-sequence of a gene encoding an acid-stable alpha-amylase from Aspergillus kawachii”, and further as EMBL: #AB008370.

The fungal alpha-amylase may also be a wild-type enzyme comprising a starch-binding domain (SBD) and an alpha-amylase catalytic domain (i.e., none-hybrid), or a variant thereof. In an embodiment the wild-type alpha-amylase is derived from a strain of Aspergillus kawachii.

### Fungal Hybrid Alpha-Amylase

In a preferred embodiment the fungal acid alpha-amylase is a hybrid alpha-amylase. Preferred examples of fungal hybrid alpha-amylases include the ones disclosed in WO 2005/003311 or U.S. Patent Publication no. 2005/0054071 (Novozymes) or U.S. patent application No. 60/638,614 (Novozymes) which are hereby incorporated by reference. A hybrid alpha-amylase may comprise an alpha-amylase catalytic domain (CD) and a carbohydrate-binding domain/module (CBM), such as a starch binding domain, and optional a linker.

Specific examples of contemplated hybrid alpha-amylases include those disclosed in Tables 1 to 5 of the examples in co-pending U.S. patent application No. 60/638,614) including Fungamyl variant with catalytic domain JA118 and Athelia rolfsii SBD (SEQ ID NO. 2 herein and SEQ ID NO: 100 in U.S. 60/638,614): Rhizomucor pusillus alpha-amylase with Athelia rolfsii AMG linker and SBD (SEQ ID NO: 3 herein and SEQ ID NO: 101 in U.S. 60/638,614), Rhizomucor pusillus alpha-amylase with Aspergillus niger glucoamylase linker and SBD (which is disclosed in Table 5 as a combination of amino acid sequences SEQ ID NO: 20 SEQ ID NO: 72 and SEQ ID NO: 96 in U.S. application Ser. No. 11/316,535 and further as SEQ ID NO: 13 herein) or as V039 in Table 5 in WO 2006/069290, and Meripilus giganteus alpha-amylase with Athena rolfsii glucoamylase linker and SBD (SEQ ID NO: 4 herein and SEQ ID NO: 102 in U.S. 60/638,614). Other specifically contemplated hybrid alpha-amylases are any of the ones listed in Tables 3, 4, 5, and 6 in Example 4 in U.S. application Ser. No. 11/316,535 and WO 2006/069290 (hereby incorporated by reference).

Other specific examples of contemplated hybrid alpha-amylases include those disclosed in U.S. Patent Publication no. 2005/0064071, including those disclosed in Table 3 on page 15, such as Aspergillus niger alpha-amylase with Aspergillus kawachii linker and starch binding domain.

Contemplated are also alpha-amylases which exhibit a high identity to any of above mention alpha-amylases, i.e., more than 70%, more than 75%, more than 80%, more than 85% more than 90%, more than 95%, more than 96%, more than 97%, more than 98%, more than 99% or even 100% identity to the mature enzyme sequences.

### Commercial Alpha-Amylase Products

Preferred commercial compositions comprising alpha-amylase include MYCOLASE from DSM (Gist Brocades), BAN™, TERMAMYL™ SC, FUNGAMYL™, LIQUOZYME™ X and SAN™ SUPER, SAN™ EXTRA L (Novozymes A/S) and CLARASE™ L-40,000, DEX-LO™, SPEZYME™ FRED, SPEZYME™ AA, and SPEZYME™ DELTA AA (Genencor Int.), and the acid fungal alpha-amylase sold under the trade name SP288 (available from Novozymes A/S Denmark).

### Glucoamylase

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular A. niger G1 or G2 glucoamylase (Boel et al. (1984), EMBO J. 3 (5), p. 1097-102), or variants thereof, such as those disclosed in WO 92/00381% WO 00/04136 and WO 01/04273 (from Novozymes, Denmark); the A. awamori glucoamylase disclosed in WO 84/02921, A. oryzae glucoamylase (Agric. Biol. Chem., 1991, 55 (4): 941-949), or variants or fragments thereof. Other Aspergillus glucoamylase variants include variants with enhanced thermal stability. G137A and G139A (Chen at al., 1996, Prot. Eng. 9: 499-506); D257E and D293E/Q (Chen et al. (1996) Prot. Eng. 8, 75-6582); N182 (Chen et al. (1994), Biochem. J. 301: 275-281), disulphide bonds, A246C (Fierobe et al., 1996, Biochemistry, 35: 8698-8704, and introduction of Pro residues in position A435 and S436 (Li et al., 1997, Protein Eng. 10: 1199-1204.

Other glucoamylases include Athelia rolfsii (previously denoted Corticium rolfsii) glucoamylase (see U.S. Pat. No. 4,727,026 and (Nagasaka, Y. et al., 1998, “Purification and properties of the raw-starch-degrading glucoamylases from Corticium rolfsii, Appl Microbiol Biotechnol 50, 323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215).

Bacterial glucoamylases contemplated include glucoamylases from the genus Clostridium, in particular C. thermoamyolyticum (EP 135,138), and C. thermohydrosulfuricum (WO 86/01831) and Trametes cingulata disclosed in co-pending U.S. provisional application No. 60/650,612 filed Feb. 7, 2005, or co-pending International application no. PCT/US05/46724 (published as WO 20080669289)) and disclosed in SEQ ID NO: 5 herein (which are hereby incorporated by reference).

Also hybrid glucoamylase are contemplated according to the invention. Examples include the hybrid glucoamylases disclosed in WO 2005/045018. Specific examples include the hybrid glucoamylase disclosed in Tables 1 and 4 of Example 1 (which hybrids are hereby incorporated by reference).

Contemplated are also glucoamylases which exhibit a high identity to any of above mention glucoamylases, i.e., more than 70%, more than 75%, more than 80%, more than 85% more than 90%, more than 95%, more than 96%, more than 97%, more than 98%, more than 99% or even 100% identity to the mature enzymes sequences.

Commercially available compositions comprising glucoamylase include AMG 200L, AMG 300 L; SAN™ SUPER, SAN™ EXTRA L, SPIRIZYME™ PLUS, SPIRIZYME™ FUEL, SPIRIZYME™ B4U and AMG™ E (from Novozymes A/S), OPTIDEX™ 300 (from Genencor Int.); AMIGASE™ and AMIGASE™ PLUS (form DSM); G-ZYME™ G900, G-ZYME™ and G990 ZR (from Genencor Int.).

Glucoamylases may in an embodiment be added in an amount of 0.02-20 AGU/g DS, preferably 0.1-10 AGU/g DS, especially between 1-5 AGU/g DS, such as 0.5 AGU/g DS.

### Proteases

In an embodiment of the invention a protease may be present during saccharification and/or fermentation.

In a preferred embodiment the protease is an acid protease of microbial origin, preferably of fungal or bacterial origin.

Suitable proteases include microbial proteases, such as fungal and bacterial proteases. Preferred proteases are acidic proteases, i.e., proteases characterized by the ability to hydrolyze proteins under acidic conditions below pH 7.

Contemplated acid fungal proteases include fungal proteases derived from Aspergillus, Mucor, Rhizopus, Candida, Coriolus, Etdothia, Enthomophtra, Irpex, Penicilium, Sclerotiumand Torulopsis. Especially contemplated are proteases derived from Aspergillus niger (see, e.g., Koaze et al., 1964, Agr. Biol. Chem. Japan, 28: 216), Aspergillus saitoi (see, e.g., Yoshida, 1954, J. Agr. Chem. Soc. Japan, 28, 66), Aspergillus awamori (Hayashida et al., 1977, Agric. Biol. Chem., 42(5): 927-933, Aspergillus aculeatus (WO 95/02044), or Aspergillus oryzae, such as the pepA protease; and acidic proteases from Mucor pusillus or Mucor miehei.

Contemplated are also neutral or alkaline proteases, such as a protease derived from a strain of Bacillus. A particular protease contemplated for the invention is derived from Bacillus amyloliquefaciens and has the sequence obtainable at Swissprot as Accession No. PO6832. Also contemplated are the proteases having at least 90% identity to amino acid sequence obtainable at Swissprot as Accession No. PO6832 such as at least 92%, at least 95%, at least 96%, at least 97%, at least 98%, or particularly at least 99% identity.

Further contemplated are the proteases having at least 90% identity to amino acid sequence disclosed as SEQ. ID. NO: 1 in the WO 2003/048353 such as at 92%, at least 95%, at least 96%, at least 97%, at least 98%, or particularly at least 99% identity.

Also contemplated are papain-like proteases such as proteases within E.C. 3.4.22.* (cysteine protease), such as EC 3.4.22.2 (papain), EC 3.4.22.6 (chymopapain), EC 3.4.22.7 (asclepain), EC 3.4.22.14 (actimidain), EC 3.4.22.15 (cathepsin L), EC 3.4.22.25 (glycyl endopeptidase) and EC 3.4.22.30 (caricain).

Proteases may be added in the amounts of 0.1-1000 AU/kg dm, preferably 1-100 AU/kg DS and most preferably 5-25 AU/kg DS.

### Additional Ingredients

Additional ingredients may be present during saccharification and/or fermentation to increase the effectiveness of the process of the invention. For instance, nutrients (e.g., fermentation organism micronutrients), antibiotics, salts (e.g., zinc or magnesium salts), other enzymes such as phytase, pullulanase, protease, beta-amylase, cellulase, glucoamylase, and hemicellulase, or a mixture thereof.

### Recovery of Fermentation Product

The fermentation product, such as ethanol, may optionally be recovered after fermentation. The recovery may be performed by any conventional manner such as, e.g., distillation.

### Process of Producing a Fermentation Product

In a further aspect the invention relates to a process for producing a fermentation product from starch-containing material comprising the steps of:

(a) liquefying starch-containing material in the presence of an alpha-amylase,

(b) saccharifying the liquefied material obtained in step (a) at a temperature in the range from 20-60° C. in the presence of:
i) from 0.001-50 AGU/g DS, preferably 0.01 to 10 AGU/g DS alpha-glucosidase activity more than the native amount of endogenous alpha-glucosidase present in the starch-containing material, and optionally
ii) from above 0 (zero) to 10 FAU-F/g DS of alpha-amylase activity,

(c) fermenting using a fermenting organism.

The fermentation product, such as especially ethanol, may optionally be recovered after fermentation, e.g., by distillation. Suitable starch-containing starting materials are listed in the section “Starch-containing materials”-section above. Examples of contemplated starch-containing material can be found the “Starch-containing materials” section above, Especially contemplated is corn (maize), cobs, wheat, barley, rye, milo, sago, cassava, tapioca, sorghum, rice, peas, beans, sweet potatoes, or a mixture thereof preferably corn. In an embodiment the starch-containing material is plant endosperm, preferably corn endosperm.

Contemplated enzymes and amounts are listed in the “Enzymes”-section above. The fermentation is preferably carried out in the presence of yeast, preferably a strain of Saccharomyces. Suitable fermenting organisms are listed in the “Fermenting Organisms”-section below. In an embodiment step (b) and (c) are carried out simultaneously (SSF process).

In a particular embodiment, the process of the invention further comprises, prior to the step (a), the steps of:

x) reducing the particle size of the starch-containing material, preferably by milling;

y) forming a slurry comprising the starch-containing material and water.

The aqueous slurry may contain from 20-55 wt. %, preferably 26-45 wt. %, more preferably 30-40 wt. % or 30-45 wt. % starch-containing material. The slurry is heated to above the gelatinization temperature and alpha-amylase, preferably bacterial and/or acid fungal alpha-amylase, may be added to initiate liquefaction (thinning). The slurry may in an embodiment be jet-cooked to further gelatinize the slurry before being subjected to an alpha-amylase in step (a) of the invention.

More specifically liquefaction may be carried out as a three-step hot slurry process. The slurry is heated to between 60-95° C., preferably 80-85° C., and alpha-amylase is added to initiate liquefaction (thinning). Then the slurry may be jet-cooked at a temperature between 95-140° C., preferably 105-125° C., for 1-15 minutes, preferably for 3-10 minutes, especially around 5 minutes. The slurry is cooled to 60-95° C. and more alpha-amylase is added to finalize hydrolysis (secondary liquefaction). The liquefaction process is usually carried out at pH 4.5-6.5, in particular at a pH between 5 and 6. Milled and liquefied whole grains are known as mash.

The saccharification in step (b) may be carried out at conditions well known in the art. A full saccharification process may lasts up to from about 24 to about 72 hours. A pre-saccharification of typically 40-90 minutes at a temperature between 30-65° C., typically about 60° C., followed by complete saccharification during fermentation in a simultaneous saccharification and fermentation process (SSF), may be carried out. Saccharification is typically carried out at temperatures from 30-65° C., typically around 60° C., and at a pH between 4 and 5, normally at about pH 4.5.

The most widely used process in ethanol production is the simultaneous saccharification and fermentation (SSF) process, in which there is no holding stage for the saccharification, meaning that fermenting organism, such as yeast, and enzyme(s) may be added together.

As also mentioned above the higher amounts of alpha-glucosidase may in one embodiment be provided by using a plant material modified to contain higher amount of alpha-glucosidase compared to starch-containing plant material derived from unmodified plants. In such case the invention relates to process for producing a fermentation product from starch-containing material derived from a modified plant comprising:

(a) liquefying starch-containing material in the presence of an alpha-amylase;

(b) saccharifying starch-containing material below the initial gelatinization temperature in the presence of:
i) alpha-glucosidase activity, and optionally
ii) from 0 (zero) to 10 FAU-F/g DS of alpha-amylase activity,

(c) fermenting using a fermenting organism,

wherein the amount of alpha-glucosidase in step (a) is higher that the native amount of endogenous alpha-glucosidase in corresponding unmodified starch-containing plant material.

In an embodiment the fermentation product is recovered after fermentation.

In a preferred embodiment the alpha-glucosidase activity amount is in the range from 0.001-50 AGU/g DS, preferably 0.01 to 10 AGU/g DS above the native amount of endogenous alpha-glucosidase in corresponding unmodified starch-containing material. The modified starch-containing plant material may be derived from a transgenic plant. A transgenic plant may be prepared using techniques well known in the art. Examples are described in the “Expression of alpha-glucosidase in transgenic plants” section above. According to the invention the transgenic plant material has a higher amount of endogenous alpha-glucosidase activity compared to the native amount of endogenous alpha-glucosidase in corresponding unmodified starch-containing plant material.

All enzymes used according to the invention include any of the ones mentioned in the “Enzymes” section above. For instance, the alpha-glucosidase may be any alpha-glucosidase, preferably those described in the “Alpha-glucosidase”-section above and in the amounts described in that section. Also the alpha-amylase may be any alpha-amylase, preferably those described in the “Alpha-amylase”-section above and in the amounts described in that section. In a preferred embodiment saccharification step (b) and fermentation step (c) are carried simultaneously. The process conditions may be as mentioned above.

In an embodiment other enzyme activities, such as protease, glucoamylase, cellulase, hemicellulase, beta-amylase and phytase activity, or mixtures thereof, may be present using saccharification. In an embodiment the sugar concentration is kept at a level below about 6 wt. %, preferably 3 wt. %, during saccharification and fermentation, especially below 0.25 wt. %.

### COMPOSITION OF THE INVENTION

In this aspect the invention relates to a composition comprising an alpha-glucosidase and an alpha-amylase.

The alpha-glucosidase may be derived from a microorganism, preferably bacteria or a fungus, or a plant. In a preferred embodiment the alpha-glucosidase is of plant origin, especially corn alpha-glucosidase. Examples of alpha-glucosidase are given above in the “Alpha-glucosidase”-section. The alpha-amylase may be derived from fungal or bacterial alpha-amylases, preferably an acidic alpha-amylase Examples of alpha-amylase are given above in the “Alpha-Amylase”-section. In a preferred embodiment the alpha-amylase comprises one or more starch binding domains (SBDs). The composition of the invention may also contain other ingredients including nutrients, antibiotics, salts or enzymes such as phytase, pullulanase, protease, beta-amylase, cellulase, glucoamylase and hemicellulase, or a mixture thereof.

### Use of a Composition of the Invention

In the final aspect the invention relates to the use of a composition for saccharification or simultaneous saccharification and fermentation. The composition may also be used in a fermentation product process, preferably for producing ethanol. The composition may also be used in a process of the invention.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

### Materials & Methods

### Enzymes:

Alpha-Amylase A: Hybrid alpha-amylase disclosed in SEQ ID NO: 13 herein consisting of Rhizomucor pusillus alpha-amylase (SEQ ID NO: 7 herein) with Aspergillus niger glucoamylase linker (SEQ ID NO: 9 herein) and 880 (SEQ ID NO: 11 herein) disclosed as V039 in Table 5 in co-pending International Application no. PCT/US05/46725 (published as WO 2006/069290).

Rice alpha-glucosidase (Sigma Cat. No. G9259-100UN).

Corn alpha-glucosidase prepared as described in Examples 1 and 2.

Bacillus stearothermophilus alpha-glucosidase is available from Sigma (Sigma cat. No, G3651).

Yeast alpha-glucosidase from Saccharomyces cereviceae is available from Sigma (Sigma Cat. No. G0660)

Glucoamylase TC: Glucoamylase derived from Trametes cingulata disclosed in SEQ ID NO: 2 in WO 2006/069289 and available from Novozymes A/S.

Glucoamylase AN: Glucoamylase derived from Aspergillus niger disclosed in Boel et al., 1984, EMBO J., 3 (5) 1097-1102 and available from Novozymes A/S.

Glucoamylase SF: Glucoamylase derived from Talaromyces emersonii, disclosed as SEQ ID NO: 7 in WO 99/28448 and available from Novozymes A/S Denmark,

### Yeast

RED STAR™ available from Red Star/Lesaffre, USA

### Determination of Identity

In context of the present invention the degree of identity between two amino acid sequences is determined by computer programs GAP provided in the GCG program package (Program Manual for the Wisconsin Package, Version 84 August 1994, Genetics Computer Group, 575 Science Drive, Madison, Wis., USA 53711) (Needleman, S. B. and Wunsch, C. D., 1970, Journal of Molecular Biology, 48: 443-453. The following settings for polypeptide sequence comparison are used: GAP creation penalty of 3.0 and GAP extension penalty of 0.1.

### Alpha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C. +/−0.05; 0.0003 M Ca²⁺; and pH 5.6) dextrinizes 5260 mg starch dry substance Merck Amylum soluble.

A folder EB-SM-0009.02/01 describing this analytical method in more detail is available upon request to Novozymes A/S, Denmark, which folder is hereby included by reference.

### Acid Alpha-Amylase Activity

When used according to the present invention the activity of any acid alpha-amylase may be measured in AFAU (Acid Fungal Alpha-amylase Units). Alternatively activity of acid alpha-amylase may be measured in AAU (Acid Alpha-amylase Units).

### Acid Alpha-Amylase Units (AAU)

The acid alpha-amylase activity can be measured in AAU (Aid Alpha-amylase Units), which is an absolute method. One Acid Amylase Unit (AAU) is the quantity of enzyme converting 1 g of starch (100% of dry matter) per hour under standardized conditions into a product having a transmission at 620 nm after reaction with an iodine solution of known strength equal to the one of a color reference.

### Standard Conditions/Reaction Conditions:

Substrate: Soluble starch, Concentration approx. 20 g DS/L.
Buffer: Citrate, approx. 0.13 M, pH=4.2
Iodine solution: 40.176 g potassium iodide+0.088 g iodine/L
City water 15°-20° dH (German degree hardness)
pH: 4.2
Incubation temperature: 30° C.
Reaction time: 11 minutes

### Wavelength: 620 nm

Enzyme concentration: 0.13-0.19 AAU/mL
Enzyme working range, 0.13-0.19 AAU/mL

The starch should be Lintner starch, which is a thin-boiling starch used in the laboratory as calorimetric indicator. Lintner starch is obtained by dilute hydrochloric acid treatment of native starch so that it retains the ability to color blue with iodine. Further details can be found in EP0140410B2, which disclosure is hereby included by reference.

### Determination of FAU-F

FAU(F) Fungal alpha-amylase Units (Fungamyl) is measured relative to an enzyme standard of a declared strength. The assay substrate is 4,6-ethylidene(G₇)-p-nitrophenyl(G₁)-alpha, D-maltoheptaoside (ethylidene-G₇-PNP).

Reaction conditions

| Temperature | 37° | C. |
|---|---|---|
| pH | 7.15 | |
| Wavelength | 405 | nm |
| Reaction time | 5 | min |
| Measuring time | 2 | min |

A folder (EB-SM-216.02) describing this standard method in more detail is available on request from Novozymes A/S, Denmark, which folder is hereby incorporated by reference.

### Acid Alpha-Amylase Activity (AFAU)

Acid alpha-amylase activity may be measured in AFAU (Acid Fungal Alpha-amylase Units) which are determined relative to an enzyme standard, 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

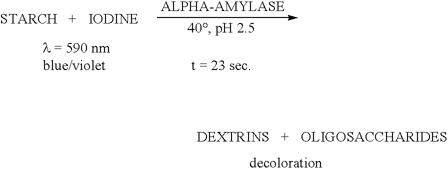

Standard Conditions/Reaction Conditions:

Substrate: Soluble starch, approx. 0.17 g/L

Buffer: Citrate, approx. 0.03 M

Iodine (I2): 0.03 g/L

CaCl2: 1.85 mm

pH: 2.50±0.05

Incubation temperature: 40° C.

Reaction time, 23 seconds

Wavelength: 590 nm

Enzyme concentration: 0.025 AFAU/mL

Enzyme working range. 0.01-0.04 AFAU/mL

A folder EB-SM-0259.02/01 describing this analytical method in more detail is available upon request to Novozymes A/S, Denmark, which folder is hereby incorporated by reference.

### Glucoamylase and Alpha-Glucosidase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

AMG Incubation:

Substrate: maltose 23.2 mM

Buffer: acetate 0.1 M

pH: 4.30±0.05

Incubation temperature: 37° C.±1

Reaction time. 5 minutes

Enzyme working range: 0.5-4.0 AGU/mL

Color Reaction:

GlucDH: 430 U/L

Mutarotase: 9 U/L

NAD: 0.21 mM

Buffer-phosphate 0.12 M; 0.15 M NaCl

pH: 7.60±0.05

Incubation temperature: 37° C.±1

Reaction time: 5 minutes

Wavelength: 340 nm

A folder (E1-SM-0131.02/01) describing this analytical method in more detail is available on request from Novozymes A/S, Denmark, which folder is hereby incorporated by reference.

### Proteolytic Activity (AU)

The proteolytic activity may be determined with denatured hemoglobin as substrate. In the Anson-Hemoglobin method for the determination of proteolytic activity denatured hemoglobin is digested, and the undigested hemoglobin is precipitated with trichloroacetic acid (TCA). The amount of TOA soluble product is determined with phenol reagent, which gives a blue color with tyrosine and tryptophan.

One Anson Unit (AU) is defined as the amount of enzyme which under standard conditions (i.e., 25° C., pH 7.5 and 10 min. reaction time) digests hemoglobin at an initial rate such that there is liberated per minute an amount of TCA soluble product which gives the same color with phenol reagent as one milliequivalent of tyrosine.

A folder AF 4/5 describing the analytical method in more detail is available upon request to Novozymes A/S, Denmark, which folder is hereby incorporated by reference.

### Determination of pH Optimum of Alpha-Glucosidase

The activity (AGU assay) of alpha-glucosidase is determined at various pH's between 2 and 8 at 37° C. for 1 hour.

### EXAMPLES

### Example 1

### Isolation of Alpha-Glucosidase from Ground Corn

Whole corn grain was milled through a 1 mm screen to four and immediately suspended in ice-chilled buffer consisting of 20 mM sodium acetate/acetic acid (pH 4.5), 0.1 mM dithiothreitol (DTT) and 0.1 mM phenylmethylsulfonylfluoride (PMSF). The suspension was swiftly stirred at 4° C. for 3 hours. The equilibrated corn slurry was centrifuged at 3700 rpm for 30 minutes. The supernatant was collected as corn enzyme extraction. The corn enzyme extract was filtered through a filter paper to remove non-soluble substances and then further filtered through a 0.45 micrometer filter to remove fine particles. The clarified solution was concentrated by an ultra-filtration unit equipped with a 10,000 Dalton cut-off membrane cassette (Pellicon XL, from Milfipore Corp). The concentrated solution was kept at 4° C. for overnight. After settling overnights the concentrated solution was centrifuged at 3700 rpm for 30 minutes. Activity assay of the collected supernatant gave an alpha-glucosidase activity of 4.0 AGU/ml and very low alpha-amylase activity. The solution was further concentrated using an Amicon ultra-filtration unit fitted with a 10,000 Dalton cut-off membrane. The concentrated sample was dialyzed using a dialysis membrane with 25,000 Dalton cut-off size (Spectrum Laboratories, Inc. CA, USA, VCAT# 132554) against the buffer for 20 hours. The final concentrated enzyme extract has 16.8 AGU/ml of alpha-glucosidase activity and was subjected to further purification.

### Example 2

### Purification of Corn Alpha-Glucosidase

All steps were carried out at 2-5° C. and the buffer used was 20 mM sodium acetate/acetic acid (pH 4.0) containing 0.1 mM DTT and 0.1 mM PMSF throughout the purification process, unless stated otherwise.

Step 1: Solid ammonium sulfate of 0-20% saturation (106 g/L) was added to the corn enzyme extract obtained in Example 1. The mixture solution was stirred for 2 hours. Supernatant was recovered after centrifugation (15,000 rpm for 10 minutes) and was added with solid ammonium sulfate of 20-75% saturation (349 g/L) and stirred for 2 hours. After centrifugation, the precipitate was dissolved with 40 ml of buffer and dialyzed against the buffer for 20 hours.

Step 2: The dialyzed sample was applied to a CM-Toyopearl column previously equilibrated with the buffer. After washing the column with the buffer, the alpha-glucosidase was eluted with a linear gradient of 0-0.75 M sodium chloride. The active fractions were combined and concentrated by Amicon™ ultra-filtration unit.

Step 3: The concentrated enzyme was applied to a Sepharose 12 HR 10/30 equilibrated with the buffer containing 0.2 M sodium chloride and eluted with the same buffer. The active fractions were used for further study.

### Example 3

### Determination of Native Endogenous Alpha-Glucosidase Activity in Corn Grain

A 10% (w/v) of corn grain flour was suspended in 30 mM of sodium acetate/acetic acid (buffer pH was 4.0) and mixed for 1 hour. While stirring continuously, 1 ml of corn slurry is taken out and added to a reaction tube containing 1 ml of 2% (w/v) maltose. The reaction mixture was incubated with shaking at 37° C. for 30-60 minutes. The reaction was stopped by 20 micro liters of 40% (v/v) sulfuric acid (H₂SO₄) and centrifuge at 3700 rpm for 10 minutes, Supernatant was collected, filtered through a 0.45 micrometer filter and then injected into a HPLC. Agilent™ 1100 HPLC system was coupled with RI detector and used to determine maltose and glucose The separation column was Aminex™ HPX-87H ion exclusion column (300 mm×7.5 mm) from BioRad™.

### Results

The activity of native endogenous corn alpha-glucosidase was found to be between 1-2 AGU/g DS determined using HPLC.

### Example 4

### Performance of Corn Alpha-Glucosidase Combined with Alpha-Amylase A on Corn Starch

Three different enzyme dosages of Alpha-Amylase A were combined with purified corn alpha-glucosidase. The dosages of Alpha-Amylase A were 0.024, 0.047 and 0.094 FAU-F/g DS, respectively, and the dosages for corn alpha-glucosidase were 0, 0.1, 0.2 and 0.4 AGU/g DS, respectively. The enzyme combination was added into 3% (w/v) corn starch slurry previously equilibrated in 50 mM sodium acetate/acetic acid, pH 4.0, containing 0.025% NaN₃ and 1 mM CaCl₂ at 32, with shaking. Oligosaccharides produced after 19 hours reaction was analyzed on a HPLC. The HPLC preparation consisted of stopping the reaction by addition of 50 microliters of 40% H₂SO₄, centrifuging, and filtering through a 0.45 micrometer filter. Samples were stored at 4° C. prior to analysis. Agilent 1100 HPLC system coupled with RI detector was used to determine ethanol and sugars. The separation column was Aminex™ HPX-87H ion exclusion column (300 mm×7.8 mm) from BioRad™.

### Results

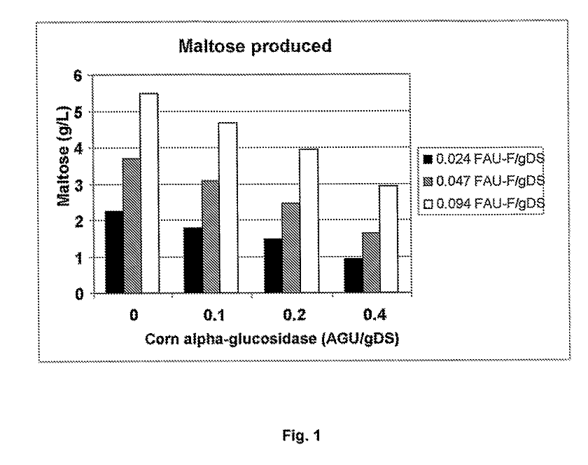

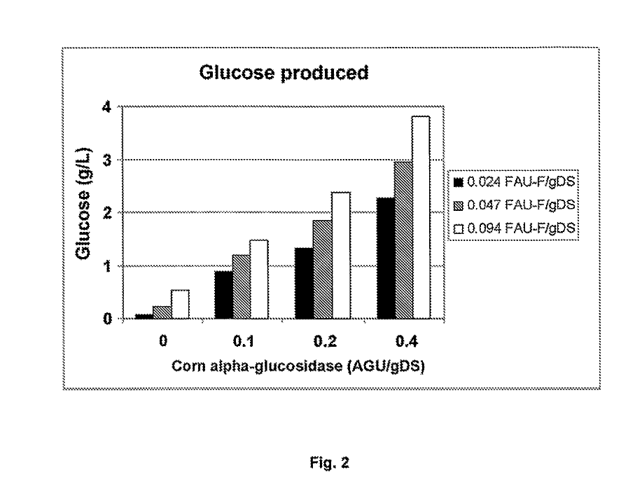

Different combinations of corn alpha-glucosidase and Alpha-Amylase A dosages were tested for corn starch hydrolysis. The tests showed that more oligosaccharides, especially maltose, were generated from corn starch with increasing alpha-amylase dosages. The produced oligosacchaddes, especially maltose, were hydrolyzed further to glucose by addition of corn alpha-glucosidase. Increased glucose production was found with increased corn alpha-glucosidase dosages. The results of the tests are shown in FIGS. 1 and 2.

### Example 5

### Performance of Corn Alpha-Glucosidase Combined with Alpha-Amylase A in One-Step Simultaneous Saccharification and Fermentation (SSF)

All treatments were evaluated via mini-scale fermentations. 410 g of ground yellow dent corn (with an average particle size around 0.5 mm) was added to 590 g tap water. This mixture was supplemented with 3.0 ml 1 g/L penicillin and 1 g of urea. The pH of this slurry was adjusted to 4.5 with 5 N NaOH (initial pH, before adjustment was about 3.8). DS level was determined to be 35 wt. %. Approximately 5 g of this slurry was added to 20 ml vials. Each vial was dosed with the appropriate amount of enzyme followed by addition of 200 micro liters yeast propagate/5 g slurry. Actual enzyme dosages were based on the exact weight of corn slurry in each vial. Purified corn alpha-glucosidase and alpha-amylase were used in this study. Vials were incubated at 32° C., 9 replicate fermentations of each treatment were run. Three replicates were selected for 24 hours, 48 hours and 70 hours time point analysis. Vials were vortexed at 24, 48 and 70 hours and analyzed by HPLC. The HPLC preparation consisted of stopping the reaction by addition of 50 micro liters of 40% H₂SO₄, centrifuging, and filtering through a 0.45 micrometer filter. Samples were stored at 4° C. prior to analysis. Agilent™ 1100 HPLC system coupled with RI detector was used to determine oligosaccharides. The separation column was aminex HPX-87H ion exclusion column (300 mm×7.8 mm) from BioRad™.

### Results

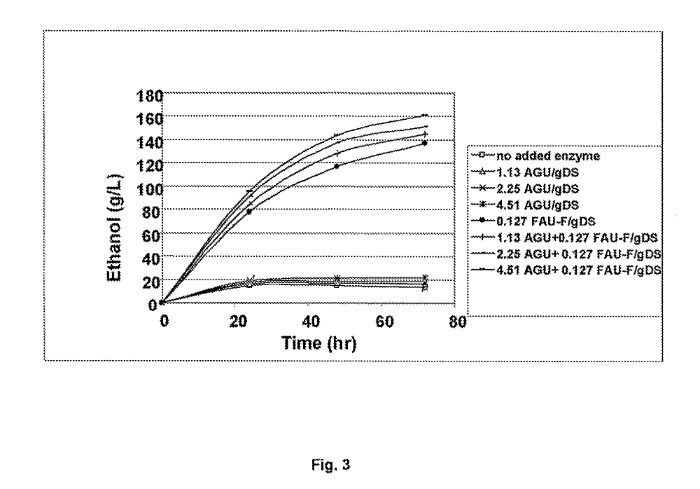

The results are shown in Table 1 below and FIG. 3. Increased amount of corn alpha-glucosidase results in increased ethanol yield. Higher ethanol yields were obtained when corn alpha-glucosidase was used in combination of Alpha-Amylase A.

TABLE 1

| | Alpha- | Alpha- | | | |
|---|---|---|---|---|---|
| | glucosidase | Amylase A | Ethanol Yield (g/l) | | |
| Trial | (AGU/ | (FAU- | 24 | 48 | 70 |
| No | g DS) | F/g DS) | hours | hours | hours |
| 1 | 0.00 | 0.000 | 14.6 | 15.0 | 13.6 |
| 2 | 1.13 | 0.000 | 16.2 | 17.0 | 15.9 |
| 3 | 2.25 | 0.000 | 17.5 | 18.1 | 18.6 |
| 4 | 4.51 | 0.000 | 18.9 | 20.8 | 21.8 |
| 5 | 0.00 | 0.127 | 77.8 | 117.0 | 137.0 |
| 6 | 1.13 | 0.127 | 84.2 | 128.1 | 144.6 |
| 7 | 2.25 | 0.127 | 91.1 | 137.2 | 151.5 |
| 8 | 4.51 | 0.127 | 95.7 | 143.7 | 161.1 |

### Example 6

### Comparison of Corn Alpha-Glucosidase to Rice Alpha-Glucosidase

The pH stability, temperature stability and ethanol stability of corn alpha-glucosidase was compared to rice alpha-glucosidase (Sigma Cat. No. G9259-100UN).

### pH Stability

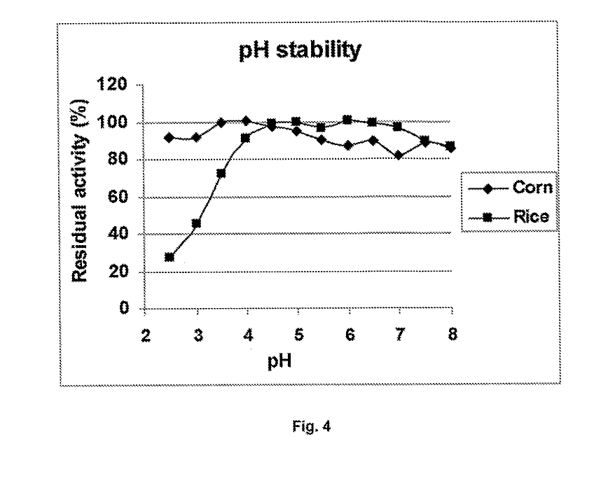

The residual activity of corn and rice alpha-glucosidase was determined after incubated at various pHs between 2 and 8 at 37° C. for 1 hours. The result is shown in FIG. 4.

### Temperature Stability

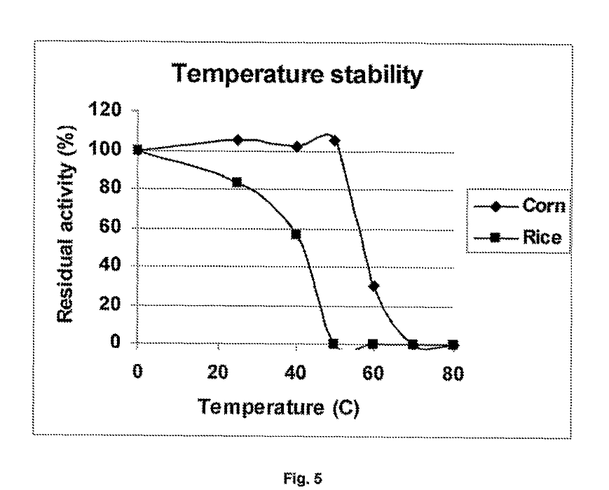

The residual activity of corn and rice alpha-glucosidase was determined after incubated at various temperatures at pH 4.0 for 2 hours. The result is shown in FIG. 5.

### Ethanol Stability

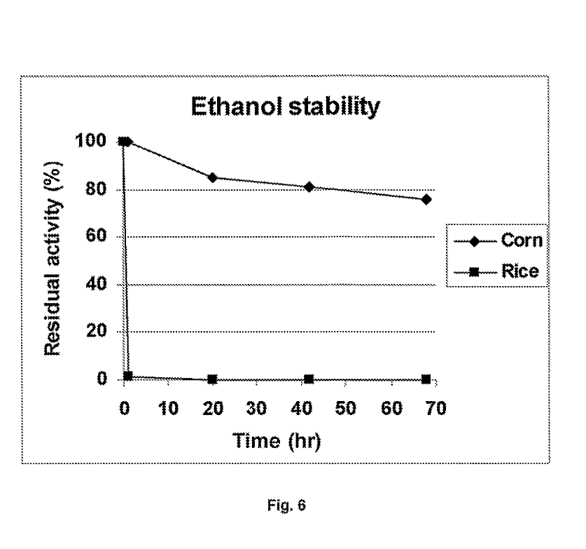

Each enzyme (0.2 mg/ml; 40 micro grams per test) was added into the buffer mixture (0.2 M NaAc, pH 4.0) containing 20 vol. % final concentration of ethanol in a tightly sealed Ependorf tube and incubated at 32° C. An appropriate amount of the mixture was taken out periodically (approximately 1 micro gram enzyme) and assayed for residual activity with maltose as substrate, Glucose produced was determined by glucose assay kit (Wako Pure Chemicals Japan) and ethanol concentration was 0.7% during assay which does not disturb the assay analysis. The result is shown in FIG. 6.

### Example 7

### Performance of Alpha-Glucosidases from Corn, Bacillus or Yeast combined with Alpha-Amylase A and Glucoamylase TC in a One-Step Simultaneous Saccharification and Fermentation Process (SSF)

All treatments were evaluated via mini-scale fermentations. 410 g of ground yellow dent corn (with an average particle size around 0.5 mm) was added to 590 g tap water. This mixture was supplemented with 3.0 ml 1 g/L penicillin and 1 g of urea. The pH of this slurry was adjusted to 4.5 with 5 N NaOH (initial pH, before adjustment was about 3.8). DS level was determined to be 35 wt. %. Approximately 5 g of this slurry was added to 20 ml vials. Each vial was dosed with the appropriate amount of enzyme followed by addition of 200 micro liters yeast propagate/5 g slurry. Actual enzyme dosages were based on the exact weight of corn slurry in each vial. Purified corn, Bacillus stearothermophilus (Sigma G3651) or yeast (Sigma G060), alpha-glucosidase and alpha-amylase and glucoamylase were used in this study. Vials were incubated at 32° C. 9 replicate fermentations of each treatment were run. Three replicates were selected for 24 hours, 48 hours and 70 hours time point analysis. Vials were vortexed at 24, 48 and 70 hours and analyzed by HPLC. The HPLC preparation consisted of stopping the reaction by addition of 50 micro liters of 40% H2SO₄, centrifuging, and filtering through a 0.45 micrometer filter. Samples were stored at 4° C. prior to analysis. Agilent™ 1100 HPLC system coupled with RI detector was used to determine oligosaccharides. The separation column was aminex HPX-87H on exclusion column (300 mm×7.8 mm) from BioRad™.

Enzyme dosages used is show in below table:

| | FIG. | FIG. | FIG. | FIG. |
|---|---|---|---|---|
| Enzyme/FIG. # | 7 | 8 | 9 | 10 |
| Corn alpha-glucosidase (AGU/g DS) | 2.6 | 2.6 | 2.6 | — |
| Bacillus stearothermophilus alpha- | — | — | — | 10 |
| glucosidase (units/g DS) | | | | |
| Yeast alpha-glucosidase (units/g DS) | — | — | — | 25 |
| Alpha-amylase A (FAU-F/g DS | 0.127 | 0.057 | 0.057 | 0.057 |
| Glucoamylase TC (AGU/g DS) | 0.34 | — | — | 0.34 |
| Glucoamyiase AN (AGU/g DS) | — | 1.0 | — | — |
| Glucoamyiase SF (AGU/g DS) | — | — | 1.68 | — |

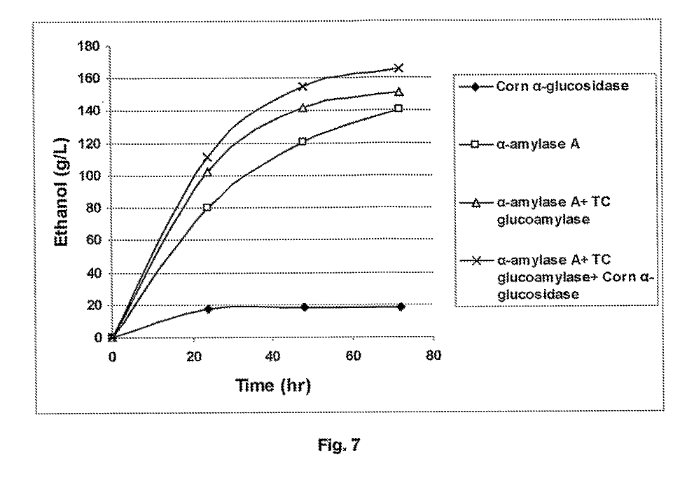

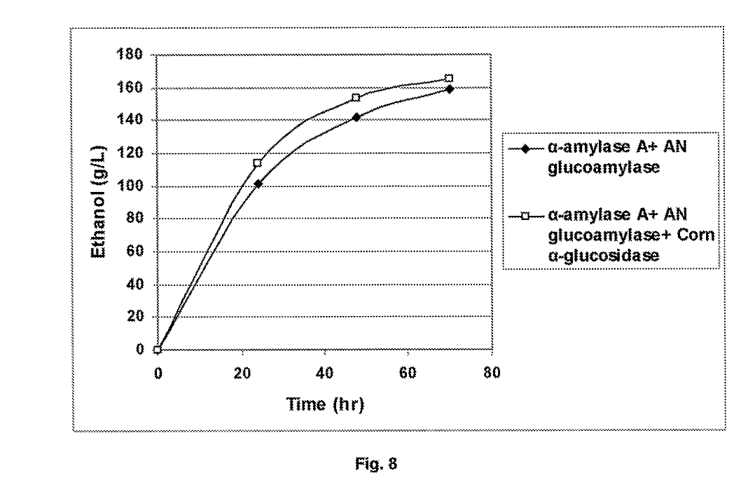

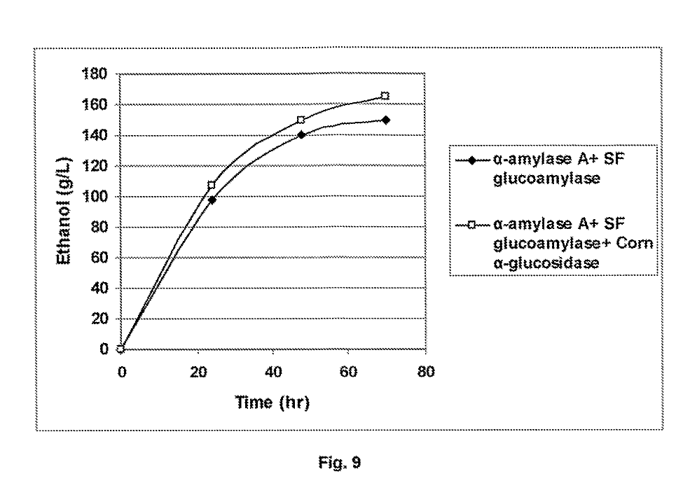

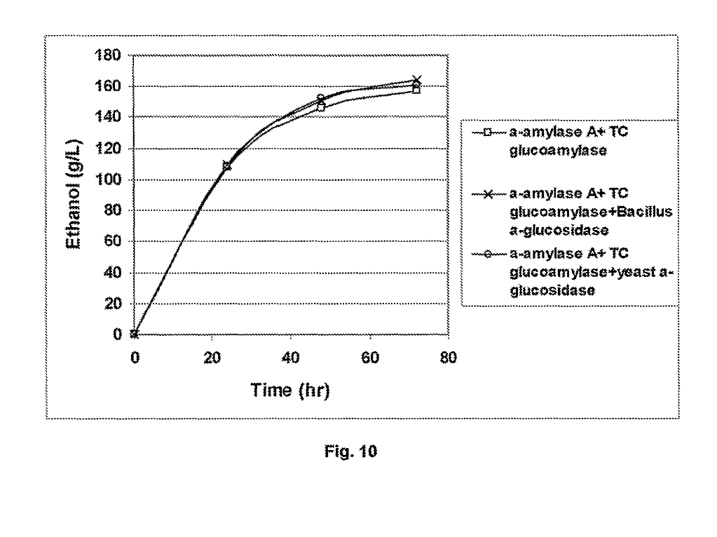

The results are shown in FIGS. 7-10.

### SUMMARY PARAGRAPHS

The present invention is defined in the claims and accompanying description. For convenience, other aspects of the present invention are presented herein by way of numbered paragraphs.

1. A process for producing a fermentation product from starch-containing material comprising:

(a) saccharifying starch-containing material below the initial gelatinization temperature in the presence of
i) from 0.001-50 AGU/g DS, preferably 0.01 to 10 AGU/g DS alpha-glucosidase activity more than the native amount of endogenous alpha-glucosidase present in the starch-containing material, and
ii) from above 0 (zero) to 10 FAU-F/g DS of alpha-amylase activity,

(b) fermenting using a fermenting organism.

2. A process for producing a fermentation product from starch-containing material derived from a modified plant comprising:

(a) saccharifying starch-containing material below the initial gelatinization temperature in the presence of
i) alpha-glucosidase activity, and
ii) from above 0 (zero) to 10 FAU-F/g DS of alpha-amylase activity,

(b) fermenting using a fermenting organism,

wherein the amount of alpha-glucosidase in step (a) is higher that the native amount of endogenous alpha-glucosidase in corresponding unmodified starch-containing plant material.
3. The process of paragraph 2, wherein the alpha-glucosidase activity amount is from 0.001-50 AGU/g DS, preferably 0.01 to 10 AGU/g DS above the native amount of endogenous alpha-glucosidase in corresponding unmodified starch-containing material
4. The process of paragraph 2 or 3, wherein the modified starch-containing plant material is derived from transgenic plant material.
5. The process of paragraph 4, wherein the transgenic plant material has a higher amount of endogenous alpha-glucosidase activity compared to the native amount of endogenous alpha-glucosidase in corresponding unmodified starch-containing plant material.
6. The process of any of paragraphs 1-5, wherein the fermentation product is recovered after fermentation.
7. The process of any of paragraphs 1-6 wherein steps (a) and (b) are carried out sequentially or simultaneously (i.e., one-step fermentation)
8. The process of any of paragraphs 1-7, wherein the alpha-glucosidase activity comes from an alpha-glucosidase derived from a microorganism, preferably bacteria, fungal organism, or a plant.
9. The process of any of paragraphs 1-8, wherein the alpha-glucosidase is an acid alpha-glucosidase.
10. The process of any of paragraphs 1-9, wherein the alpha-glucosidase is plant alpha-glucosidase, preferably derived from a plant selected from the group consisting of corn (maize), cobs, wheat, barley, rye, milo, sago, cassava, tapioca, sorghum, rice, peas, beans, sweet potatoes, or a mixture thereof, preferably corn.
11. The process of any of paragraphs 1-9, wherein the fungal alpha-glucosidase is derived from yeast, preferably a strain of Candida spa, preferably Candida edax, or a strain of Saccharomyces sp. preferably Saccharomyces cerevisiae.
12. The process of any of paragraphs 1-9, wherein the fungal alpha-glucosidase is derived from a filamentous fungus, preferably a strain of Aspergillus, preferably Aspergillus nidulans, Aspergillus niger, Aspergillus oryzae, or Aspergillus fumigatus.
13. The process of any of paragraphs 1-9, wherein the alpha-glucosidase is derived from bacteria, preferably a strain of Bacillus sp., preferably Bacillus stearothermophilus.
14. The process of any of paragraphs 1-13, wherein the alpha-glucosidase activity level is from 0.1 to B AGU/g DS, preferably 1 to 6 AGU/g DS alpha-glucosidase activity, higher than the native amount of endogenous alpha-glucosidase present in the starch-containing material before saccharification.
15. The process of any of paragraphs 1-14, wherein the total amount of endogenous alpha-glucosidase present during saccharification and/or fermentation in from above 2 to 12 AGU/g DS alpha-glucosidase activity, preferable from 3 to 10 AGU/9 DS, especially 4 to 8 AGU/g DS alpha-glucosidase activity.
16. The process of any of paragraphs 1-15, wherein alpha-amylase is present during saccharification step (a) or simultaneous saccharification and fermentation step (a) and (b) in from 0.01 to 3 FAU-F/g DS alpha-amylase activity, preferably from 0.06 to 0.2 FAU-F/g DS alpha-amylase activity.
17. The process of any of paragraphs 1-16, wherein the starch-containing material is plant material selected from the corn (maize), cobs, wheat, barley, rye, milo, sag, cassava, tapioca, sorghum, rice, peas, beans, sweet potatoes, or a mixture thereof, preferably corn.
18. The process of any of paragraphs 1-165 wherein the starch-containing material is derived from transgenic plant material with a higher amount of alpha-glucosidase activity compared to corresponding unmodified plant material, such as transgenic corn material.
19. The process of any of paragraphs 1-18, wherein the starch-containing material is plant endosperm, preferably corn endosperm.
20. The process of any of paragraphs 1-195 wherein the starch-containing material is granular starch.
21. The process of any of paragraphs 1-20, wherein the process is carried out at a pH in the range between 3 and 7, preferably from 3 to 6, or more preferably from 3.5 to 5.0.
22. The process of any of paragraphs 1-21, wherein the dry solid content (DS) ties in the range from 20-55 wt. % r, preferably 25-45 wt. %, more preferably 30-40 wt. % or 30-45 wt. %.
23. The process of any of paragraphs 1-22, wherein the sugar concentration is kept at a level below about 6 wt. %, preferably 3 wt. %, during saccharification and fermentation, especially below 0.25 wt. %.
24. The process of any of paragraphs 1-23, wherein a slurry comprising starch-containing material reduced in particle size and water, is prepared before step (a).
25. The process of any of paragraphs 1-24, wherein the starch-containing material is prepared by reducing the particle size of the starch-containing material, preferably by milling, such that at least 50% of the starch-containing material has a particle size of 0.1-0.5 mm.
26. The process of any of paragraphs 1-25, wherein the starch-containing material is dry or wet milled.
27. The process of any of paragraphs 1-26, wherein the starch-containing plant material is reduced in particle size with particle size emulsion technology.
28. The process of any of paragraphs 1-27, wherein the fermentation is carried out for 30 to 150 hours, preferably 48 to 96 hours.
29. The process of any of paragraphs 1-28, wherein the temperature during fermentation in step (b) or simultaneous saccharification and fermentation in steps (a) and (b) is between 25° C. and 400° C. preferably between 28° C. and 36° C., such as between 28° C. and 35° C., such as between 28° C. and 34° C., such as around 32° C.
30. The process of any of paragraphs 1-29, wherein a protease is present during saccharification and/or fermentation.
31. The process of any of paragraphs 1-30, wherein backset is added before and/or during saccharification and/or fermentation.
32. The process of any of paragraphs 1-32, wherein a nitrogen source is added to before and/or during saccharification and/or fermentation.
33. The process of any of paragraphs 1-32, wherein the alpha-amylase activity is derived from fungal or bacterial alpha-amylases, preferably an acidic alpha-amylase.
34. The process of any of paragraphs 1-33, wherein the alpha-amylase activity comes from a wild-type alpha-amylase or a variant thereof.
35. The process of any of paragraphs 1-34, wherein the alpha-amylase activity comes from a fungal alpha-amylase, preferably derived from the genus Aspergillus, especially a strain of Aspergillus niger, Aspergillus oryzae, Aspergillus awamori, or Aspergillus kawachii.
36. The process of any of paragraphs 1-35, wherein the alpha-amylase activity comes from a wild-type alpha-amylase or variant thereof comprising one or more starch binding domains (SBDs).
37. The process of any of paragraphs 1-36, wherein the alpha-amylase activity comes from Aspergillus kawachii alpha-amylase.
38. The process of any of paragraphs 1-37, wherein the alpha-amylase activity comes from alpha-amylase derived from a strain of the genus Rhizomucor, preferably a strain the Rhizomucor pusillus, or the genus Meripilus, preferably a strain of Meripilus giganteus.
39. The process of any of paragraphs 1-38, wherein the alpha-amylase activity comes from a hybrid alpha-amylase comprising one or more starch binding domains (SBDs).
40. The process of any of paragraphs 1-39, wherein the alpha-amylase activity comes from a hybrid alpha-amylase selected from the group of Fungamyl variant with catalytic domain JA118 and Athelia rolfsii SD (SEQ ID NO: 2 herein), Rhizomucor pusillus alpha-amylase with Athelia rolfsii glucoamylase linker and SBD (SEQ ID NO: 3 herein), Meripilus giganteus alpha-amylase with Athelia rolfsii glucoamylase linker and SBD (SEQ ID NO: 4 herein) or Rhizomucor pusillus alpha-amylase with Aspergillus niger glucoamylase linker and SBD (SEQ ID NO: 13).
41. The process of any of paragraphs 1-40, wherein the alpha-amylase activity comes from a hybrid alpha-amylase comprising Aspergillus niger alpha-amylase with Aspergillus kawachii linker and Aspergillus kawachii starch binding domain (SBD).
42. The process of any of paragraphs 1-41, wherein the alpha-amylase activity comes from a bacterial alpha-amylase, preferably derived a strain of the genus Bacillus, preferably a strain of Bacillus licheniformis, Bacillus amyloliquefaciens, Bacillus stearothermophilus, or Bacillus subtilis.
43. The process of paragraph 42, wherein the bacterial alpha-amylase is derived from a strain of Bacillus stearothermophilus, having the mutations I181*+G182*, preferably I181*+G182*+N 193F compared to the wild type amino acid sequence set forth in SEQ to NO: 3 in WO 99/19467.
44. The process of paragraph 43, wherein the bacterial alpha-amylase is a hybrid alpha-amylase comprising the 445 C-terminal amino acid residues of the Bacillus licheniformis alpha-amylase set forth in SEQ ID NO:4 in WO 99/19467 and the 37 N-terminal amino acid residues of the alpha-amylase derived from Bacillus amyloliquefaciens set forth in SEQ ID NO: 5 in WO 99/19467% having the substitution G48A+T49I+G107A+H156Y+A181T+N910F+I201F+A209V+Q264S (using SEQ ID NO: 4 numbering in WO 99/19467).
45. The process of any of paragraphs 1-44, wherein one or more enzymes selected from the group consisting of glucoamylase, phytase, pullulanase, beta-amylase, cellulase, and hemicellulase, or a mixture thereof, is (are) present during saccharification or fermentation, or simultaneous saccharification and fermentation (SSF).
46. The process of paragraph 45, wherein the glucoamylase is derived from the genus Aspergillus, preferably a strain of Aspergillus niger, Aspergillus oryzae, Aspergillus awamori, or the genus Athelia, preferably a strain of Athelia rolfsii, the genus Talaromyces, preferably a strain the Talaromyces emersonii, or the genus Rhizopus, such as a strain of Rhizopus nivius, or of the genus Humicola, preferably a strain of Humicola grisea var. thermoidea, or a strain of the genus Trametes, preferably a strain of Trametes cingulate.
47. The process of paragraph 45 or 46, wherein glucoamylase is present in an amount of 0.001 to 10 AGU/g DS preferably from 0.01 to 5 AGU/g DS, especially 0.1 to 0.5 AGU/g DS.
48. The process of any of paragraphs 1-47, wherein the fermentation product is an alcohol, preferably ethanol, especially fuel ethanol, portable ethanol and/or industrial ethanol.
49. A process for producing a fermentation product from starch-containing material comprising the steps of:

(a) liquefying starch-containing material in the presence of an alpha-amylase;

(b) saccharifying the liquefied material obtained in step (a) at a temperature in the range from 20-60° C. in the presence of:
i) from 0.001-50 AGU/g DS, preferably 0.01 to 10 AGU/g DS alpha-glucosidase activity more than the native amount of endogenous alpha-glucosidase present in the starch-containing material, and optionally
ii) from 0 (zero) to 10 FAU-F/g DS of alpha-amylase activity,

(c) fermenting using a fermenting organism.

50. A process for producing a fermentation product from starch-containing material derived from a modified plant comprising:

(a) liquefying starch-containing Material in the presence of an alpha-amylase;

(b) saccharifying starch-containing material below the initial gelatinization temperature in the presence of:
i) alpha-glucosidase activity, and optionally
ii) from 0 (zero) to 10 FAU-F/g DS of alpha-amylase activity,

(c) fermenting using a fermenting organism,

wherein the amount of alpha-glucosidase in step (a) is higher that the native amount of endogenous alpha-glucosidase in corresponding unmodified starch-containing plant material.
51. The process of paragraph 50, wherein the alpha-glucosidase amount is from 0.001-50 AGU/g DS, preferably 0.01 to 10 AGU/g DS above the native amount of endogenous alpha-glucosidase in corresponding unmodified starch-containing material
52 The process of paragraph 50 or 51, wherein the modified starch-containing plant material is derived from transgenic plant material.
53. The process of paragraph 52, wherein the transgenic plant material has a higher amount of endogenous alpha-glucosidase activity compared to the native amount of endogenous alpha-glucosidase in corresponding unmodified starch-containing plant material.
54. The process of any of paragraphs 49-53, wherein the fermentation product is recovered after fermentation.
55. The process of any of paragraphs 49-54, wherein steps (b) and (c) are carried out sequentially or simultaneously (i.e., one-step fermentation).
56. The process of any of paragraphs 49-55, wherein the fermentation product is an alcohol, preferably ethanol, especially fuel ethanol, portable ethanol and/or industrial ethanol.
57. The process of any of paragraphs 49-66, wherein the starch-containing starting material is whole grains, preferably whole corn or wheat grains.
58. The process of any of paragraphs 49-57, wherein the fermenting organism is a strain of Saccharomyces, preferably a strain of Saccharomyces cerevisiae.
59. The process of any of paragraphs 49-58, further comprising, prior to the step (a), the steps of:

x) reducing the particle size of starch-containing material;

y) forming a slurry comprising the starch-containing material and water.

60. The process of paragraph 59, wherein the slurry is heated to above the gelatinization temperature.
61. The process of paragraph 59 or 60, wherein the slurry is jet-cooked at a temperature between 95-140° C.) preferably 105-125° C., for 1-15 minutes) preferably for 3-10 minutes, especially around 5 minutes.
62. The process of any of paragraphs 49-61, wherein the dry solid content (DS) of the starting material lies in the range from 20-55 wt. %, preferably 25-45 wt. %, more preferably 30-40 wt. % or 30-45 wt. %.
63. The process of any of paragraphs 49-62, wherein the alpha-glucosidase activity comes from an alpha-glucosidase derived from a microorganism, preferably bacteria, fungal organism, or a plant.
64. The process of any of paragraphs 49-63, wherein the alpha-glucosidase is an acid alpha-glucosidase.
65. The process of any of paragraphs 49-64, wherein the alpha-glucosidase is a plant alpha-glucosidase, preferably derived from a plant selected from the group consisting of corn (maize), cobs, wheat, barley, rye, milo, sago, cassava, tapioca, sorghum rice, peas, beans, sweet potatoes, or a mixture thereof, preferably corn.
66. The process of any of paragraphs 49-65, wherein the fungal alpha-glucosidase is derived from yeast, preferably a strain of Candida sp., preferably Candida edex, or a strain of Saccharomyces sp. preferably Saccharomyces cerevisiae.
67. The process of any of paragraphs 49-66, wherein the fungal alpha-glucosidase is derived from a filamentous fungus, preferably a strain of Aspergillus, preferably Aspergillus nidulans, Aspergillus niger, Aspergillus oryzae, or Aspergillus fumigatus.
68. The process of any of paragraphs 49-67, wherein the alpha-glucosidase is derived from bacteria, preferably a strain of Bacillus sp., preferably Bacillus stearothermophilus.
69. The process of any of paragraphs 49-68, wherein the alpha-glucosidase activity level is from 0.1 to 8 AGU/g DS, preferably 1 to 6 AGU/g DS alpha-glucosidase activity, higher than the native amount of endogenous alpha-glucosidase present in the starch-containing material before saccharification.
70. The process of any of paragraphs 49-69, wherein the total amount of endogenous alpha-glucosidase present during saccharification and/or fermentation in from above 2 to 12 AGU/g DS alpha-glucosidase activity, preferable from 3 to 10 AGU/g DS, especially 4 to 8 AGU/g DS alpha-glucosidase activity.
71. The process of any of paragraphs 49-70, wherein alpha-amylase is present during saccharification step (b) or simultaneous saccharification and fermentation steps (b) and (c) in from 0.01 to 3 FAU-F/g DS alpha-amylase activity, preferably from 0.05 to 0.2 FAU-F/g DS alpha-amylase activity.
72. The process of any of paragraphs 49-71, wherein the starch-containing material is plant material selected from the corn (maize), cobs, wheat, barley, rye, milo, sago, cassava, tapioca, sorghum, rice, peas, beans, sweet potatoes, or a mixture thereof, preferably corn.
73. The process of any of paragraphs 49-72, wherein the starch-containing material is derived from transgenic plant material with a higher amount of alpha-glucosidase activity compared to corresponding unmodified plant material, such as transgenic corn material.
74. The process of any of paragraphs 49-73, wherein the starch-containing material is plant endosperm, preferably corn endosperm.
75. The process of any of paragraphs 49-74) wherein the process is carried out at a pH in the range between 3 and 7, preferably from 3 to 6, or more preferably from 3.6 to 5.0.
76. The process of any of paragraphs 49-75, wherein the sugar concentration is kept at a level below about 6 wt. %, preferably 3 wt. %, during saccharification and fermentation, especially below 0.25 wt. %.
77. The process of any of paragraphs 49-76, wherein the fermentation is carried out for 30 to 150 hours, preferably 48 to 96 hours.
78. The process of any of paragraphs 49-77, wherein the temperature during fermentation in step (c) or simultaneous saccharification and fermentation in steps (b) and (c) is between 25° C. and 40° C., preferably between 28° C. and 36° C., such as between 28° C. and 356° C. such as between 280° C. and 34° C., such as around 32° C.
79. The process of any of paragraphs 49-78, wherein further a protease is present during saccharification and/or fermentation.
80. The process of any of paragraphs 49-79, wherein backset is added before and/or during saccharification and/or fermentation.
81. The process of any of paragraphs 49-80, wherein a nitrogen source is added to before and/or during saccharification and/or fermentation.
82. The process of any of paragraphs 49-81, wherein the alpha-amylase activity is derived from fungal or bacterial alpha-amylases, preferably an acidic alpha-amylase.
83. The process of any of paragraphs 49-82, wherein the alpha-amylase activity comes from a wild-type alpha-amylase or a variant thereof.
84. The process of any of paragraphs 49-83, wherein the alpha-amylase activity comes from a fungal alpha-amylase, preferably derived from the genus Aspergillus, especially a strain of Aspergillus niger, Aspergillus oryzae, Aspergillus awamori, or Aspergillus kawachii.
85. The process of any of paragraphs 49-84, wherein the alpha-amylase activity comes from a wild-type alpha-amylase or variant thereof comprising one or more starch binding domains (SBDs).
86. The process of any of paragraphs 49-85, wherein the alpha-amylase activity comes from Aspergillus kawachii alpha-amylase.
87. The process of any of paragraphs 49-86, wherein the alpha-amylase activity comes from alpha-amylase derived from a strain of the genus Rhizomucor, preferably a strain the Rhizomucor pusillus, or the genus Meripilus, preferably a strain of Meripilus giganteus.
88. The process of any of paragraphs 49-87, wherein the alpha-amylase activity comes from a hybrid alpha-amylase comprising one or more starch binding domains (SBDs).
89. The process of any of paragraphs 49-88, wherein the alpha-amylase activity comes from a hybrid alpha-amylase selected from the group of Fungamyl variant with catalytic domain JA118 and Athelia rolfsii S38 (SEQ ID NO: 2 herein), Rhizomucor pusillus alpha-amylase with Athelia rolfsii glucoamylase linker and SBD (SEQ ID NO: 3 herein), Meripilus giganteus alpha-amylase with Athelia rolfsii glucoamylase linker and SBD (SEQ ID NO: 4 herein) or Rhizomucor Pusillus alpha-amylase with Aspergillus niger glucoamylase linker and SBD (SEQ ID NO: 13).
90. The process of any of paragraphs 49-90, wherein the alpha-amylase activity comes from a hybrid alpha-amylase comprising Aspergillus niger alpha-amylase with Aspergillus kawachii linker and Aspergillus kawachii starch binding domain (SBD).
91. The process of any of paragraphs 49-90, wherein the alpha-amylase activity comes from a bacterial alpha-amylase, preferably derived a strain of the genus Bacillus, preferably a strain of Bacillus licheniformis, Bacillus amyloliquefaciens, Bacillus stearothermophilus, or Bacillus subtilis.
92. The process of paragraph 91, wherein the bacterial alpha-amylase is derived from a strain of Bacillus stearothermophilus, having the mutations I181*+B182*, preferably I181*+G182*, +N193F compared to the wild type amino acid sequence set forth in SEQ ID NO: 3 in WO 099/19467.
93. The process of paragraph 91 wherein the bacterial alpha-amylase is a hybrid alpha-amylase comprising the 445 C-terminal amino acid residues of the Bacillus licheniformis alpha-amylase set forth in SEQ ID NO: 4 in WO 99/1947 and the 37 N-terminal amino acid residues of the alpha-amylase derived from Bacillus amyloliquefaciens set forth in SEQ ID NO: 5 in WO 99/19467, having the substitution 348A+T491+G107A+H156Y+A181T+N190F+I201F+A209V+Q264S (using SEQ ID NO: 4 numbering in WO 99/19467).
94. The process of any of paragraphs 49-93, wherein one or more enzymes selected from the group consisting of glucoamylase, phytase, pullulanase, beta-amylase, cellulase, and hemicellulase, or a mixture thereof, is (are) present during saccharification or fermentation, or simultaneous saccharification and fermentation (SSF).
95. The process of paragraph 94, wherein the glucoamylase is derived from the genus Aspergillus, preferably a strain of Aspergillus niger, Aspergillus oryzae, Aspergillus awamori, or the genus Athelia, preferably a strain of Athelia rolfsii, the genus Talaromyces, preferably a strain the Talaromyces emersonii, or the genus Rhizopus, such as a strain of Rhizopus nivius, or of the genus Humicola, preferably a strain of Humicola grisea var. thermoidea, or a strain of the genus Trametes, preferably a strain of Trametes cingulata.
96. The process of paragraph 94 or 95, wherein glucoamylase is present in an amount of 0.001 to 10 AGU/g DS, preferably from 0.01 to 5 AGU/g DS, especially 0.01 to 0.5 AGU/g DS.
97. A composition comprising an alpha-glucosidase and an alpha-amylase.
98. The composition of paragraph 97, wherein the alpha-glucosidase is derived from a microorganism, preferably bacteria or a fungus, or a plant.
99. The composition of paragraph 97 or 98, wherein the alpha-glucosidase is plant alpha-glucosidase, preferably derived from a plant selected from the group consisting of corn (maize), cobs, wheat, barley, rye, milo, sago, cassava, tapioca, sorghum, rice, peas, beans, sweet potatoes, or a mixture thereof, preferably corn.
100. The composition of any of paragraphs 97-99, wherein the alpha-glucosidase is derived from yeast, preferably a strain of Candida sp., preferably Candida edax, or a strain of Saccharomyces sp. preferably Saccharomyces cerevisiae.
101. The composition of any of paragraphs 97-100, wherein the alpha-glucosidase is derived from a filamentous fungus, preferably a strain of Aspergillus, preferably Aspergillus nidulans, Aspergillus niger, Aspergillus oryzae, or Aspergillus fumigatus.
102. The composition of any of paragraphs 97-101, wherein the alpha-glucosidase is derived from bacteria, preferably a strain of Bacillus sp., preferably Bacillus stearothermophilus.
103. The composition of any of paragraphs 97-102, wherein the alpha-amylase is derived from fungal or bacterial alpha-amylases, preferably an acidic alpha-amylase.
104. The composition of any of paragraphs 97-103, wherein the alpha-amylase is a wild-type alpha-amylase or a variant thereof.
105. The composition of any of paragraphs 97-104, wherein the alpha-amylase is a fungal alpha-amylase, preferably derived from the genus Aspergillus, especially a strain of Aspergillus niger, Aspergillus oryzae, Aspergillus awamori, or Aspergillus kawachii.
106. The composition of any of paragraphs 97-105, wherein the alpha-amylase is a wild-type alpha-amylase or variant thereof comprising one or more starch binding domains (SBDs).
107. The composition of any of paragraphs 97-106, wherein the alpha-amylase is an Aspergillus kawachii alpha-amylase.
108. The composition of any of paragraphs 97-107, wherein the alpha-amylase is an alpha-amylase derived from a strain of the genus Rhizomucor, preferably a strain the Rhizomucor pusillus, or the genus Meripilus, preferably a strain of Meripilus giganteus.
109. The composition of any of paragraphs 97-108, wherein the alphaamylase is a hybrid alpha-amylase comprising one or more starch binding domains (SBDs).
110. The composition of any of paragraphs 97-109, wherein the alpha-amylase is a hybrid alpha-amylase selected from the group of Fungamyl variant with catalytic domain JA118 and Athelia rolfsii 88 (SEQ ID NO: 2 herein), Rhizomucor pusillus alpha-amylase with Athelia rolfsii glucoamylase linker and SBD (SEQ ID NO: 3 herein), Meripilus giganteus alpha-amylase with Athelia rolfsii glucoamylase linker and SBD (SEQ ID NO: 4 herein) or Rhizomucor pusillus alpha-amylase with Aspergillus niger glucoamylase linker and SBD (SEQ ID NO: 13).
111. The composition of any of paragraphs 97-110, wherein the alpha-amylase is a hybrid alpha-amylase comprising Aspergillus niger alpha-amylase with Aspergillus kawachii linker and Aspergillus kawachii starch binding domain (SB).
112. The composition of any of paragraphs 97-111, wherein the alpha-amylase is a bacterial alpha-amylase, preferably derived a strain of the genus Bacillus, preferably a strain of Bacillus licheniformis, Bacillus amyloliquefaciens, Bacillus stearothermophilus, or Bacillus subtilis.
113. The composition of any of paragraphs 97-112 wherein the bacterial alpha-amylase is derived from a strain of Bacillus stearothermophilus, having the mutations I181*+G182*, preferably I181*+G182*, +N193F compared to the wild type amino acid sequence set forth in SEQ ID NO: 3 in WO 99/19467.
114. The composition of any of paragraphs 97-113, wherein the bacterial alpha-amylase is a hybrid alpha-amylase comprising the 445 C-terminal amino acid residues of the Bacillus licheniformis alpha-amylase set forth in SEQ ID NO:4 in WO 99/19467 and the 37 N-terminal amino acid residues of the alpha-amylase derived from Bacillus amyloliquefaciens set forth in SEQ ID NO, 5 in WO 99/19467, having the substitution G48A+T49I+G107A+H156Y+A181T+N190F+1201F+A209V+Q264S (using SEQ ID NO: 4 numbering in WO 99/19467).
115 The composition of any of paragraphs 97-114, wherein the composition further comprises one or more components selected from the group of nutrients, antibiotics, salts or enzymes such as phytase, glucoamylase, pullulanase, protease, beta-amylase, cellulase, and hemicellulase, or a mixture thereof.
116. The composition of paragraph 115, wherein the glucoamylase is a fungal glucoamylase.
117. The composition of paragraph 115 or 116 wherein the glucoamylase is derived from the genus Aspergillus, preferably a strain of Aspergillus niger, Aspergillus oryzae, Aspergillus awamori, or the genus Athelia, preferably a strain of Athelia rolfsii, the genus Talaromyces, preferably a strain the Talaromyces emersonii or the genus Rhizopus, such as a strain of Rhizopus nivius, or of the genus Humicola, preferably a strain of Humicola grisea var. thermoidea, or a strain of the genus Trametes, preferably a strain of Trametes cingulata.
118. Use of a composition of any of paragraphs 97-117% for simultaneous saccharification and fermentation.
119. Use of a composition of any of paragraphs 97-117 for ethanol production.
120. Use of a composition of any of paragraphs 97-117 in a process of any of paragraphs 1 to 96.

1-37. (canceled)

38. A process for producing a fermentation product from starch-containing material comprising:
(a) saccharifying starch-containing material below the initial gelatinization temperature in the presence of:
i) from 0.001-50 AGU/g DS, preferably 0.01 to 10 AGU/g DS alpha-glucosidase activity more than the native amount of endogenous alpha-glucosidase present in the starch-containing material, and
ii) from above 0 (zero) to 10 FAU-F/g DS of alpha-amylase activity,
(b) fermenting using a fermenting organism.

39. The process of claim 38, wherein the alpha-glucosidase activity amount is from 0.001-50 AGU/g DS.

40. The process of claim 38, wherein the modified starch-containing plant material is derived from transgenic plant material.

41. The process of claim 40, wherein the transgenic plant material has a higher amount of endogenous alpha-glucosidase activity compared to the native amount of endogenous alpha-glucosidase in corresponding unmodified starch-containing plant material.

42. The process of claim 38, further comprising recovering the fermentation product after fermentation.

43. The process of claim 38, wherein steps (a) and (b) are carried out sequentially or simultaneously (i.e., one-step fermentation)

44. The process of claim 38, wherein the alpha-glucosidase activity comes from an alpha-glucosidase derived from a microorganism, preferably bacteria, fungal organism, or a plant.

45. The process of claim 38, wherein the alpha-glucosidase activity level is from 0.1 to 8 AGU/g DS higher than the native amount of endogenous alpha-glucosidase present in the starch-containing material before saccharification.

46. The process of claim 38, wherein the total amount of endogenous alpha-glucosidase present during saccharification and/or fermentation in from above 2 to 12 AGU/g DS alpha-glucosidase activity.

47. The process of claim 38, wherein alpha-amylase is present during saccharification step (a) or simultaneous saccharification and fermentation step (a) and (b) in from 0.01 to 3 FAU-F/g DS alpha-amylase activity.

48. The process of claim 38, wherein the starch-containing material is plant material selected from the corn (maize), cobs, wheat, barley, rye, milo, sago, cassava, tapioca, sorghum, rice, peas, beans, sweet potatoes, or a mixture thereof, preferably corn.

49. The process of claim 38, wherein the process is carried out at a pH in the range between 3 and 7.

50. The process of claim 38, wherein the dry solid content (DS) lies in the range from 20-55 wt. %.

51. The process of claim 38, wherein the sugar concentration is kept at a level below about 6 wt. % during saccharification and fermentation.

52. The process of claim 38, further comprising preparing a slurry comprising starch-containing material reduced in particle size and water, before step (a).

53. The process of claim 38, wherein the starch-containing material is prepared by reducing the particle size of the starch-containing material such that at least 50% of the starch-containing material has a particle size of 0.1-0.5 mm.

54. The process of claim 38, wherein the starch-containing material is dry or wet milled.

55. The process of claim 38, wherein the starch-containing plant material is reduced in particle size with particle size emulsion technology.

56. The process of claim 38, wherein the fermentation is carried out for 30 to 150 hours, preferably 48 to 96 hours.

57. A process for producing a fermentation product from starch-containing material comprising the steps of:
(a) liquefying starch-containing material in the presence of an alpha-amylase;
(b) saccharifying the liquefied material obtained in step (a) at a temperature in the range from 20-60° C. in the presence of:
i) from 0.001-50 AGU/g DS, preferably 0.01 to 10 AGU/g DS alpha-glucosidase activity more than the native amount of endogenous alpha-glucosidase present in the starch-containing material, and optionally
ii) from 0 (zero) to 10 FAU-F/g DS of alpha-amylase activity,
(c) fermenting using a fermenting organism.


SEQUENCE LISTING (13 sequences)

SEQ ID NO: 1 - DNA, 1761 bp, Artificial
  Hybrid Fungamyl variant JA118 with A. rolfsii SBD
gcaacgcctgcggactggcgatcgcaatccatttatttccttctcacggatcgatttgcaaggacggatgggtcgacgactgcgacttgtaatactgcggatcagaaatactgtggtggaacatggcagggcatcatcgacaagttggactatatccagggaatgggcttcacagccatctggatcacccccgttacagcccagctgccccagaccaccgcatatggagatgcctaccatggctactggcagcaggatatatactctctgaacgaaaactacggcactgcagatgacttgaaggcgctctcttcggcccttcatgagagggggatgtatcttatggtcgatgtggttgctaaccatatgggctatgatggaccgggtagctcagtcgattacagtgtgtttgttccgttcaattccgctagctacttccacccgttctgtttcattcaaaactggaatgatcagactcaggttgaggattgctggctaggagataacactgtctccttgcctgatctcgataccaccaaggatgtggtcaagaatgaatggtacgactgggtgggatcattggtatcgaactactccattgacggcctccgtatcgacacagtaaaacacgtccagaaggacttctggcccgggtacaacaaagccgcaggcgtgtactgtatcggcgaggtgctcgacggtgatccggcctacacttgtccctaccaggaagtcctggacggcgtactgaactaccccatttactatccactcctcaacgccttcaagtcaacctccggcagcatggacgacctctacaacatgatcaacaccgtcaaatccgactgtccagactcaacactcctgggcacattcgtcgagaaccacgacaacccacggttcgcttcttacaccaacgacatagccctcgccaagaacgtcgcagcattcatcatcctcaacgacggaatccccatcatctacgccggccaagaacagcactacgccggcggaaacgaccccgcgaaccgcgaagcaacctggctctcgggctacccgaccgacagcgagctgtacaagttaattgcctccgcgaacgcaatccggaactatgccattagcaaagatacaggattcgtgacctacaagaactggcccatctacaaagacgacacaacgatcgccatgcgcaagggcacagatgggtcgcagatcgtgactatcttgtccaacaagggtgcttcgggtgattcgtataccctctccttgagtggtgcgggttacacagccggccagcaattgacggaggtcattggctgcacgaccgtgacggttgattcgtcgggagatgtgcctgttcctatggcgggtgggctacctagggtattgtatccgactgagaagttggcaggtagcaagatctgtagtagctcgggtgctacaagcccgggtggctcctcgggtagtgtcgaggtcactttcgacgtttacgctaccacagtatatggccagaacatctatatcaccggtgatgtgagtgagctcggcaactggacacccgccaatggtgttgcactctcttctgctaactaccccacctggagtgccacgatcgctctccccgctgacacgacaatccagtacaagtatgtcaacattgacggcagcaccgtcatctgggaggatgctatcagcaatcgcgagatcacgacgcccgccagcggcacatacaccgaaaaagacacttgggatgaatcttag

SEQ ID NO: 2 - protein, 586 aa, Artificial
  Synthetic Construct
ATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGPGSSVDYSVFVPFNSASYFHPFCFIQNWNDQTQVEDCWLGDNTVSLPDLDTTKDVVKNEWYDWVGSLVSNYSIDGLRIDTVKHVQKDFWPGYNKAAGVYCIGEVLDGDPAYTCPYQEVLDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVDSSGDVPVPMAGGLPRVLYPTEKLAGSKICSSSGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 3 - protein, 558 aa, Artificial
  Hybrid alpha-amylase with Rhizomucor pusillus catalytic domain and A. rolfsii linker and SBD
SPLPQQQRYGKRATSDDWKSKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSAGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 4 - protein, 574 aa, Artificial
  Hybrid alpha-amylase with Meripilus giganteous catalytic domain with A. rolfsii linker and SBD.
RPTVFDAGADAHSLHARAPSGSKDVIIQMFEWNWDSVAAECTNFIGPAGYGFVQVSPPQETIQGAQWWTDYQPVSYTLTGKRGDRSQFANMITTCHAAGVGVIVDTIWNHMAGVDSGTGTAGSSFTHYNYPGIYQNQDFHHCGLEPGDDIVNYDNAVEVQTCELVNLADLATDTEYVRGRLAQYGNDLLSLGADGLRLDASKHIPVGDIANILSRLSRSVYITQEVIFGAGEPITPNQYTGNGDVQEFRYTSALKDAFLSSGISNLQDFENRGWVPGSGANVFVVNHDTERNGASLNNNSPSNTYVTATIFSLAHPYGTPTILSSYDGFTNTDAGAPNNNVGTCSTSGGANGWLCQHRWTAIAGMVGFRNNVGSAALNNWQAPQSQQIAFGRGALGFVAINNADSAWSTTFTTSLPDGSYCDVISGKASGSSCTGSSFTVSGGKLTATVPARSAIAVHTGQKGSGGGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 5 - protein, 561 aa, Trametes cingulata
QSSAADAYVASESPIAKAGVLANIGPSGSKSNGAKASDTPGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQASYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

SEQ ID NO: 6 - DNA, 1320 bp, Rhizomucor pusillus
  CDS (1)..(1320): Catalytic domain
agccctttgccccaacagcagcgatatggcaaaagagcaacttcggatgactggaaaggcaaggccatttatcagctgcttacagatcgatttggccgcgccgatgactcaacaagcaactgctctaatttatccaactactgtggtggtacctacgaaggcattacgaagcatcttgactacatttccggtatgggctttgatgctatctggatatcgccaattcccaagaactcggatggaggctaccacggctactgggctacagatttctaccaactaaacagcaactttggtgatgaatcccagctcaaagcgctcatccaggctgcccatgaacgtgacatgtatgttatgcttgatgtcgtagccaatcatgcaggtcccaccagcaatggctactcgggttacacattcggcgatgcaagtttatatcatcctaaatgcaccatagattacaatgatcagacgtctattgagcaatgctgggttgctgacgagttgcctgatattgacactgaaaattctgacaacgtggccattctcaacgacatcgtctccggctgggtgggtaactatagctttgacggcatccgcattgatactgtcaagcatattcgcaaggacttttggacaggctacgcagaagctgccggcgtattcgcaactggagaggtcttcaatggtgatccggcctacgttggaccttatcaaaagtacctgccatctctcatcaattacccaatgtattacgctttgaacgacgtctttgtatccaaaagcaaaggattcagccgcatcagcgaaatgctaggatcaaatcgcaatgcgtttgaggataccagcgtacttacaacgtttgtagacaaccatgacaatccgcgcttcttgaacagtcaaagcgacaaggctctcttcaagaacgctctcacatacgtactgctaggtgaaggcatcccaattgtgtattatggttctgagcaaggtttcagcggaggagcggatcctgctaaccgtgaagtgctgtggaccaccaattatgatacatccagcgatctctaccaatttatcaagacagtcaacagtgtccgcatgaaaagcaacaaggccgtctacatggatatttatgttggcgacaatgcttacgccttcaagcacggcgatgctttggttgttctcaataactatggatcaggttccacaaaccaagtcagcttcagcgttagtggcaagttcgatagcggcgcaagcctcatggatattgtcagtaacattaccaccacggtgtcctcggatggaacagtcactttcaaccttaaa

SEQ ID NO: 7 - protein, 440 aa, Rhizomucor pusillus
SPLPQQQRYGKRATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLK

SEQ ID NO: 8 - DNA, 111 bp, Aspergillus niger
  CDS (1)..(111): Linker
actggcggcaccactacgacggctacccccactggatccggcagcgtgacctcgaccagcaagaccaccgcgactgctagcaagaccagcaccagtacgtcatcaacctcc

SEQ ID NO: 9 - protein, 37 aa, Aspergillus niger
TGGTTTTATPTGSGSVTSTSKTTATASKTSTSTSSTS

SEQ ID NO: 10 - DNA, 324 bp, Aspergillus niger
  CDS (1)..(324): CBM
tgtaccactcccaccgccgtggctgtgactttcgatctgacagctaccaccacctacggcgagaacatctacctggtcggatcgatctctcagctgggtgactgggaaaccagcgacggcatagctctgagtgctgacaagtacacttccagcgacccgctctggtatgtcactgtgactctgccggctggtgagtcgtttgagtacaagtttatccgcattgagagcgatgactccgtggagtgggagagtgatcccaaccgagaatacaccgttcctcaggcgtgcggaacgtcgaccgcgacggtgactgacacctggcgg

SEQ ID NO: 11 - protein, 108 aa, Aspergillus niger [sequence omitted]

SEQ ID NO: 12 - DNA, 1755 bp, Artificial
  Hybrid Rhizomucor alpha-amylase with Aspergillus niger linker and Aspergillus niger CBM
agccctttgccccaacagcagcgatatggcaaaagagcaacttcggatgactggaaaggcaaggccatttatcagctgcttacagatcgatttggccgcgccgatgactcaacaagcaactgctctaatttatccaactactgtggtggtacctacgaaggcattacgaagcatcttgactacatttccggtatgggctttgatgctatctggatatcgccaattcccaagaactcggatggaggctaccacggctactgggctacagatttctaccaactaaacagcaactttggtgatgaatcccagctcaaagcgctcatccaggctgcccatgaacgtgacatgtatgttatgcttgatgtcgtagccaatcatgcaggtcccaccagcaatggctactcgggttacacattcggcgatgcaagtttatatcatcctaaatgcaccatagattacaatgatcagacgtctattgagcaatgctgggttgctgacgagttgcctgatattgacactgaaaattctgacaacgtggccattctcaacgacatcgtctccggctgggtgggtaactatagctttgacggcatccgcattgatactgtcaagcatattcgcaaggacttttggacaggctacgcagaagctgccggcgtattcgcaactggagaggtcttcaatggtgatccggcctacgttggaccttatcaaaagtacctgccatctctcatcaattacccaatgtattacgctttgaacgacgtctttgtatccaaaagcaaaggattcagccgcatcagcgaaatgctaggatcaaatcgcaatgcgtttgaggataccagcgtacttacaacgtttgtagacaaccatgacaatccgcgcttcttgaacagtcaaagcgacaaggctctcttcaagaacgctctcacatacgtactgctaggtgaaggcatcccaattgtgtattatggttctgagcaaggtttcagcggaggagcggatcctgctaaccgtgaagtgctgtggaccaccaattatgatacatccagcgatctctaccaatttatcaagacagtcaacagtgtccgcatgaaaagcaacaaggccgtctacatggatatttatgttggcgacaatgcttacgccttcaagcacggcgatgctttggttgttctcaataactatggatcaggttccacaaaccaagtcagcttcagcgttagtggcaagttcgatagcggcgcaagcctcatggatattgtcagtaacattaccaccacggtgtcctcggatggaacagtcactttcaaccttaaaactggcggcaccactacgacggctacccccactggatccggcagcgtgacctcgaccagcaagaccaccgcgactgctagcaagaccagcaccagtacgtcatcaacctcctgtaccactcccaccgccgtggctgtgactttcgatctgacagctaccaccacctacggcgagaacatctacctggtcggatcgatctctcagctgggtgactgggaaaccagcgacggcatagctctgagtgctgacaagtacacttccagcgacccgctctggtatgtcactgtgactctgccggctggtgagtcgtttgagtacaagtttatccgcattgagagcgatgactccgtggagtgggagagtgatcccaaccgagaatacaccgttcctcaggcgtgcggaacgtcgaccgcgacggtgactgacacctggcgg

SEQ ID NO: 13 - protein, 585 aa, Artificial
  Synthetic Construct
SPLPQQQRYGKRATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKTGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR